(19) United States
(12) Patent Application Publication
(10) Pub. No.: US 2007/0270448 A1
(43) Pub. Date: Nov. 22, 2007

(54) Methods of Using and Compositions Comprising a Jnk Inhibitor for the Treatment and Management of Asbestos-Related Diseases and Disorders

(75) Inventor: Jerome B. Zeldis, Princeton, NJ (US)

Correspondence Address: JONES DAY, 222 EAST 41ST ST, NEW YORK, NY, 10017, US

(73) Assignee: CELGENE CORPORATION, SUMMIT, NJ (US)

(21) Appl. No.: 10/578,809

(22) PCT Filed: Nov. 4, 2004
(86) PCT No.: PCT/US04/37084

§ 371 Date: Mar. 12, 2007

Related U.S. Application Data
(60) Provisional application No. 60/518,601, filed on Nov. 6, 2003.

(51) Int. Cl. A61K 31/505; A61K 31/425; A61K 31/416; A61K 31/4196; A61K 31/42; A61K 31/403; A61K 38/21; A61K 38/14; A61K 31/704; A61K 31/7072; A61K 31/4745; A61K 31/337; A61K 33/36

(52) U.S. Cl. 514/275; 514/383; 514/406; 514/410; 514/373; 514/379; 514/34; 514/283; 514/8; 424/85.4; 424/649; 424/94.61; 424/623; 514/109; 514/154; 514/49; 514/449; 514/171; 514/251; 514/492; 514/323

(57) ABSTRACT

Methods for treating, preventing and/or managing an asbestos-related disease or disorder are disclosed. Specific embodiments encompass the administration of a JNK Inhibitor, or a pharmaceutically acceptable salt, solvate, hydrate, stereoisomer, clathrate, or prodrug thereof, alone or in combination with a second active agent and/or chemotherapy, surgery, or radiation therapy. Pharmaceutical compositions, single unit dosage forms, and kits suitable for use in the methods of the invention are also disclosed.

### 1. FIELD OF INVENTION

This invention relates to methods of treating, preventing and/or managing an asbestos-related disease or disorder, which comprise the administration of a JNK Inhibitor alone or in combination with known therapeutics. The invention also relates to pharmaceutical compositions and dosing regimens. In particular, the invention encompasses the use of a JNK Inhibitor in conjunction with surgery or radiation therapy and/or other standard therapies for diseases associated with asbestos poisoning.

### 2. BACKGROUND OF THE INVENTION

### 2.1 Asbestos-Related Diseases or Disorders

Several million individuals worldwide were exposed to asbestos in the mining of ore or the manufacture and use of asbestos products. D. R. Aberle, Seminars in Roentgenology, 24 (2): 118, 1991. Given the long latency for the development of many pathological consequences of asbestos, asbestos-related diseases will likely dominate the field of occupational and environmental diseases for some time. Benign asbestos-related diseases and disorders include asbestosis, pleural effusion, pleural plaques, diffuse pleural thickening, and rounded atelectasis. C. A. Staples, Radiologic Clinics of North America, 30 (6): 1191, 1992. Malignant asbestos-related diseases include malignant pleural effusion, pleural or peritoneal mesothelioma, and bronchogenic carcinoma. Merck Index, 1999 (17^{th} ed.), 645 and 651.

Asbestosis (interstitial fibrosis) is defined as diffuse lung fibrosis due to the inhalation of asbestos fibers. C. A. Staples, Radiologic Clinics of North America, 30 (6): 1195, 1992. It is one of the major causes of occupationally related lung damage. Merck Index, 1999 (17^{th} ed.), 622. Asbestosis characteristically occurs following a latent period of 15-20 years, with a progression of disease even after exposure has ceased, but rarely occurs in the absence of pleural plaques. C. Peacock, Clinical Radiology, 55: 425, 2000. Fibrosis first arises in and around the respiratory bronchioles, predominating in the subpleural portions of the lung in the lower lobes, and then progresses centrally. C. A. Staples, Radiologic Clinics of North America, 30 (6): 1195, 1992. Asbestosis may cause an insidious onset of progressive dyspnea in addition to a dry cough. The incidence of lung cancer is increased in smokers with asbestosis, and a dose-response relationship has been observed. Merck Index, 1999 (17^{th} ed.), 623.

Another asbestos-related disorder is pleural effusion. Pleural effusions are often the earliest manifestation of asbestos-related disease. C. A. Staples, Radiologic Clinics of North America, 30 (6): 1192, 1992. People exposed to asbestos can develop an exudative pleural effusion five to 20 years after exposure. Merck Index, 1999 (17^{th} ed.), 645; C. A. Staples, Radiologic Clinics of North America, 30 (6): 1192, 1992; and C. Peacock, Clinical Radiology, 55: 427, 2000. Effusion may follow short exposure, but more often follows intermediate exposure of about 10 to 15 years. The clinical picture in benign asbestos-related pleural effusion varies from asymptomatic patients to patients with an acute episode of pleuritic chest pain and pyrexia. Id., 426. The mechanism is unknown, but it is assumed that the fibers migrate from the lungs to the pleura and induce an inflammatory response. In most people, effusions clear after three to four months, but can persist or recur over several years. Id. As the effusion resolves, many develop diffuse pleural thickening. Id.

Pleural plaques are a common manifestation of asbestos exposure, typically occurring after a latent period of approximately 20-30 years. C. A. Staples, Radiologic Clinics of North America, 30 (6): 1191, 1992; and C. Peacock, Clinical Radiology, 55: 423, 2000. Histologically, pleural plaques consist of acellular collagen bundles that form a basket-weave pattern, which almost exclusively involves the parietal pleura. C. A. Staples, Radiologic Clinics of North America, 30 (6): 1191, 1992. The precise pathogenesis of pleural plaques remains undetermined, although some have assumed that they are caused by the mechanical effect of asbestos fibers piercing the visceral pleura. C. Peacock, Clinical Radiology, 55: 425, 2000. Currently, however, the fibers are believed to be transported to the parietal pleura via lymphatic channels, where they incite an inflammatory response. Id. Plaques slowly grow over time, even after cessation of exposure, but they are not considered premalignant. Id. Calcification occurs later, often 30-40 years following exposure. Id., 424; and C. A. Staples, Radiologic Clinics of North America, 30 (6): 1191, 1992. Although there is a significant correlation between the severity of the pleural disease and that of asbestosis, pleural plaques tend to occur in isolation without any other manifestations of asbestos-related diseases. C. Peacock, Clinical Radiology, 55: 425, 2000.

Another common manifestation of asbestos exposure is diffuse pleural thickening. C. A. Staples, Radiologic Clinics of North America, 30 (6): 1193, 1992. Usually, the latent period is approximately 15 years. Diffuse pleural thickening is less specific for asbestos exposure than the presence of pleural plaques, since thickening also may be seen following TB pleuritis, hemothorax and empyema. C. Peacock, Clinical Radiology, 55: 427, 2000. The most common symptom is dyspnea. The pathogenesis is unclear, but it is believed to be due to inflammation and fibrosis of the visceral pleural lymphatics, and it has been considered an extension of parenchymal fibrosis. Id. Development of diffuse pleural thickening has a similar time-line as plaque formation. Thickening is a common concomitant finding to asbestosis, with a reported associated incidence of 10%. Id.

Another disease associated with asbestos exposure is round atelectasis, which refers to atelectatic lung adjacent to pleural thickening with characteristic in-drawing of bronchi and vessels. T. Wallace, Diagnostic Cytopathology, 8 (6): 617, 1992; C. Peacock, Clinical Radiology, 55: 429, 2000; and C. A. Staples, Radiologic Clinics of North America, 30 (6): 1193, 1992. It is also known as folded lung, pulmonary pseudotumor, pleuroma or Blesovsky syndrome. Id. The presence of the effusion has been postulated to cause passive atelectasis, with infolding of the lung resulting in invagination of the adjacent pleura. Id. This process causes tethering, which prevents reexpansion of the lung upon resolution of the effusion and which causes round atelectasis. Id. An alternative explanation is that an insult to the pleura leads to localized inflammation and fibrosis, which results in volume loss and buckling of the underlying lung. Id. The lingula is the most common site, followed by the middle and then the lower lobes, although lesions may be multiple and bilateral. Id.

Mesothelioma is a malignant pleural or peritoneal neoplasm that is usually associated with occupational exposure to asbestos. Merck Index, 1999 (17^{th} ed.), 645. The clinical latency period between asbestos exposure and mesothelioma development is typically 15-40 years. Id., 623; and C. Peacock, Clinical Radiology, 55: 427, 2000. As a result, the number of mesothelioma patients has continued to rise despite decreased asbestos production. J M W van Haarst et al., British Journal of Cancer, 86: 342, 2002. The common symptoms are chest pain, dyspnea, cough, weight loss, weakness and increased sputum production. Merck Index, 1999 (17^{th} ed.), 645. The tumor gradually encases the lungs, invades the chest wall, and produces pleural effusion in about 75% of patients. Id. The prognosis is dismal, with poor response to radial surgery, chemotherapy, or radiation therapy. Id.

The causal relationship between bronchogenic carcinoma and asbestos exposure is well accepted. Merck Index, 1999 (17^{th} ed.), 651; and D. R. Aberle, Seminars in Roentgenology, 24 (2): 124, 1991. It shows a dose response at occupational exposure levels. Id. The relative risk of lung cancer in asbestos workers increases multiplicatively with combined cigarette smoking, and asbestos-related interstitial disease is often associated with it. Id. Lung cancer has been also reported in individuals without interstitial lung disease who are exposed to asbestos. Id.

### 2.2 Conventional Treatments

The primary strategy for dealing with asbestos-related diseases or disorders is prevention, with the worldwide elimination of asbestos use and with the replacement of asbestos by safe synthetic products. No treatment for asbestosis is known to be effective. Mesothelioma is very difficult to treat, and no standard therapy for its treatment currently exists. Kaiser L R., Semin Thorac Cardiovasc Surg. October, 9 (4): 383-90, 1997. The methods of chemotherapy, radiation therapy, and surgery have all been used with little improvement in overall survival, although trimodality therapy that involves a combination of all three treatments has been shown to improve survival in selected patients. Id.

The two primary surgical interventions used to treat mesothelioma are pleurectomy and extrapleural pneumonectomy (EPP). Pleurectomy usually is a palliative procedure to relieve chest wall pain and prevent recurrent pleural effusions by stripping off the visceral and parietal pleura. C. Turton, British Journal of Hospital Medicine, 23(3): 249, 1980. EPP is an en bloc resection of the parietal and mediastinal pleura, lung, hemi-diaphragm, and ipsilateral pericardium to remove all gross disease. Sugarbaker D J, Ann Surg., 224(3):288-94, 1996. EPP is indicated for stage I tumors with no involvement of the mediastinal lymph nodes. EPP is a technically demanding surgery with significant morbidity. The surgical complications of pleurectomy and EPP include pneumonia, bronchopleural fistulae, bronchial leaks, empyema, chylothorax, respiratory insufficiency, myocardial infarction, congestive heart failure, hemorrhage, cardiac volvulus, subcutaneous emphysema, incomplete tumor removal, and vocal cord paralysis. Id.

Radiotherapy usually is palliative or adjunctive to surgery. C. Turton, British Journal of Hospital Medicine, 23(3): 249, 1980. Brachytherapy, intrapleural implantation of radioactive isotopes, delivers high-dose radiation locally to the pleural space and is used for recurrent pleural effusions. Id. Postoperative radiation therapy can prevent recurrence within chest wall incision sites. Complications of radiotherapy include nausea and vomiting, radiation hepatitis, esophagitis, myelitis, myocarditis, and pneumonitis with deterioration of pulmonary function.

Photodynamic therapy is an adjuvant treatment in patients with surgically treated pleural malignancies. P. Baas, Br. J. Cancer., 76(6): 819-26, 1997. A light-activated photosensitizing drug is instilled intrapleurally and is excited by light of a certain wavelength to produce oxygen free radicals that cause tumor necrosis. Id.

Response to chemotherapy has been disappointing because comparison of chemotherapies has been difficult. Intrapleural instillations of antibiotics such as mepacrine, thiotepa, and tetracycline have been reported to be sometimes successful. C. Turton, British Journal of Hospital Medicine 23(3): 247, 1980. Various cytotoxic drugs including mustine have been instilled into the pleural cavity. Id. Medications presently used during the treatment of mesothelioma include GM-CSF, doxorubicin, gemcitabine, cisplatin, vinblastine, adriamycin, bleomycin, hyaluronidase, methotrexate and mitomycin. J M W van Haarst et al., British Journal of Cancer, 86: 342-345, 2002. However, patients rarely obtain complete relief. Chemotherapy results in less than 20% response and has not yet been shown to improve survival in patients with mesothelioma. Id. Therefore, there remains a need for safe and effective methods of treating and managing mesothelioma and other diseases associated with exposure to asbestos.

Citation of any reference in Section 2 of this application is not an admission that the reference is prior art to the application.

### 3. SUMMARY OF THE INVENTION

This invention encompasses methods for treating, preventing and/or managing asbestos-related diseases or disorders, which comprise administering to a patient in need thereof a therapeutically or prophylactically effective amount of a JNK Inhibitor, or a pharmaceutically acceptable salt, solvate, hydrate, stereoisomer, clathrate, or prodrug thereof.

Another embodiment of the invention encompasses the use of one or more JNK Inhibitors in combination with other therapeutics typically used to treat or prevent asbestos-related diseases or disorders such as, but not limited to, anti-cancer agents, antibiotics, anti-inflammatory agents, cytokines, steroids, immunomodulatory agents, immunosuppressive agents, and other known therapeutics.

Yet another embodiment of the invention encompasses the use of one or more JNK Inhibitors in combination with conventional therapies used to treat, prevent or manage asbestos-related diseases or disorders including, but not limited to, chemotherapy, surgery, radiation therapy and photodynamic therapy.

The invention further encompasses pharmaceutical compositions, single unit dosage forms, and kits suitable for use in treating, preventing and/or managing asbestos-related diseases or disorders, which comprise one or more JNK Inhibitors, or a pharmaceutically acceptable salt, solvate, hydrate, stereoisomer, clathrate, or prodrug thereof, and one or more additional active agents.

### 3.1 DEFINITIONS

As used herein, the term “patient” means an animal (e.g., cow, horse, sheep, pig, chicken, turkey, quail, cat, dog, mouse, rat, rabbit or guinea pig), preferably a mammal such as a non-primate or a primate (e.g., monkey or human), most preferably a human.

“Alkyl” means a saturated straight chain or branched non-cyclic hydrocarbon having from 1 to 10 carbon atoms. “Lower alkyl” means alkyl, as defined above, having from 1 to 4 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl; while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

An “alkenyl group” or “alkylidene” mean a straight chain or branched non-cyclic hydrocarbon having from 2 to 10 carbon atoms and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like. An alkenyl group can be unsubstituted or substituted. A “cyclic alkylidene” is a ring having from 3 to 8 carbon atoms and including at least one carbon-carbon double bond, wherein the ring can have from 1 to 3 heteroatoms.

An “alkynyl group” means a straight chain or branched non-cyclic hydrocarbon having from 2 to 10 carbon atoms and including at least one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl, and the like. An alkynyl group can be unsubstituted or substituted.

The terms “Halogen” and “Halo” mean fluorine, chlorine, bromine or iodine.

“Haloalkyl” means an alkyl group, wherein alkyl is defined above, substituted with one or more halogen atoms.

“Keto” means a carbonyl group (i.e.,C═O).

“Acyl” means an —C(O)alkyl group, wherein alkyl is defined above, including —C(O)CH₃, —C(O)CH₂CH₃, —C(O)(CH₂)₂CH₃, —C(O)(CH₂)₃CH₃, —C(O)(CH₂)₄CH₃, —C(O)(CH₂)₅CH₃, and the like.

“Acyloxy” means an —OC(O)alkyl group, wherein alkyl is defined above, including —OC(O)CH₃, —OC(O)CH₂CH₃, —OC(O)(CH₂)₂CH₃, —OC(O)(CH₂)₃CH₃, —OC(O)(CH₂)₄CH₃, —OC(O)(CH₂)₅CH₃, and the like.

“Ester” means and —C(O)Oalkyl group, wherein alkyl is defined above, including —C(O)OCH₃, —C(O)OCH₂CH₃, —C(O)O(CH₂)₂CH₃, —C(O)O(CH₂)₃CH₃, —C(O)O(CH₂)₄CH₃, —C(O)O(CH₂)₅CH₃, and the like.

“Alkoxy” means —O-(alkyl), wherein alkyl is defined above, including —OCH₃, —OCH₂CH₃, —O(CH₂)₂CH₃, —O(CH₂)₃CH₃, —O(CH₂)₄CH₃, —O(CH₂)₅CH₃, and the like. “Lower alkoxy” means —O-(lower alkyl), wherein lower alkyl is as described above.

“Alkoxyalkoxy” means —O-(alkyl)-O-(alkyl), wherein each alkyl is independently an alkyl group defined above, including —OCH₂OCH₃, —OCH₂CH₂OCH₃, —OCH₂CH₂OCH₂CH₃, and the like.

“Alkoxycarbonyl” means —C(═O)O-(alkyl), wherein alkyl is defined above, including —C(═O)O—CH₃, —C(═O)O—CH₂CH₃, —C(═O)O—(CH₂)₂CH₃, —C(═O)O—(CH₂)₃CH₃, —C(═O)O—(CH₂)₄CH₃, —C(═O)O—(CH₂)₅CH₃, and the like.

“Alkoxycarbonylalkyl” means -(alkyl)-C(═O)O-(alkyl), wherein each alkyl is independently defined above, including —CH₂—C(═O)O—CH₃, —CH₂—C(═O)O—CH₂CH₃, —CH₂—C(═O)O—(CH₂)₂CH₃, —CH₂—C(═O)O—(CH₂)₃CH₃, —CH₂—C(═O)O—(CH₂)₄CH₃, —CH₂—C(═O)O—(CH₂)₅CH₃, and the like.

“Alkoxyalkyl” means -(alkyl)-O-(alkyl), wherein each alkyl is independently an alkyl group defined above, including —CH₂OCH₃, —CH₂OCH₂CH₃, —(CH₂)₂OCH₂CH₃, —(CH₂)₂O(CH₂)₂CH₃, and the like.

“Aryl” means a carbocyclic aromatic group containing from 5 to 10 ring atoms. Representative examples include, but are not limited to, phenyl, tolyl, anthracenyl, fluorenyl, indenyl, azulenyl, pyridinyl and naphthyl, as well as benzo-fused carbocyclic moieties including 5,6,7,8-tetrahydronaphthyl. A carbocyclic aromatic group can be unsubstituted or substituted. In one embodiment, the carbocyclic aromatic group is a phenyl group.

“Aryloxy” means —o-aryl group, wherein aryl is as defined above. An aryloxy group can be unsubstituted or substituted. In one embodiment, the aryl ring of an aryloxy group is a phenyl group

“Arylalkyl” means -(alkyl)-(aryl), wherein alkyl and aryl are as defined above, including —(CH₂)phenyl, —(CH₂)₂phenyl, —(CH₂)₃phenyl, —CH(phenyl)₂, —CH(phenyl)₃, —(CH₂)tolyl, —(CH₂)anthracenyl, —(CH₂)fluorenyl, —(CH₂)indenyl, —(CH₂)azulenyl, —(CH₂)pyridinyl, —(CH₂)naphthyl, and the like.

“Arylalkyloxy” means —O-(alkyl)-(aryl), wherein alkyl and aryl are defined above, including —O—(CH₂)₂phenyl, —O—(CH₂)₃phenyl, —O—CH(phenyl)₂, —O—H(phenyl)₃, —O—(CH₂)tolyl, —O—(CH₂)anthracenyl, —O—(CH₂)fluorenyl, —O—(CH₂)indenyl, —O—(CH₂)azulenyl, —O—(CH₂)pyridinyl, —O—(CH₂)naphthyl, and the like.

“Aryloxyalkyl” means -(alkyl)-O-(aryl), wherein alkyl and aryl are defined above, including —CH₂—O-(phenyl), —(CH₂)₂—O-phenyl, —(CH₂)₃—O-phenyl, —(CH₂)—O-tolyl, —(CH₂)—O-anthracenyl, —(CH₂)—O-fluorenyl, —(CH₂)—O-indenyl, —(CH₂)—O-azulenyl, —(CH₂)—O-pyridinyl, —(CH₂)—O-naphthyl, and the like.

“Cycloalkyl” means a monocyclic or polycyclic saturated ring having carbon and hydrogen atoms and having no carbon-carbon multiple bonds. Examples of cycloalkyl groups include, but are not limited to, (C₃-C₇)cycloalkyl groups, including cyclopropyl, cyclobutyl, cyclopentyl, cyclohexyl, and cycloheptyl, and saturated cyclic and bicyclic terpenes. A cycloalkyl group can be unsubstituted or substituted. In one embodiment, the cycloalkyl group is a monocyclic ring or bicyclic ring.

“Cycloalkyloxy” means —O-(cycloalkyl), wherein cycloalkyl is defined above, including —O-cyclopropyl, —O-cyclobutyl, —O-cyclopentyl, —-cyclohexyl, —O-cycloheptyl and the like.

“Cycloalkylalkyloxy” means —O-(alkyl)-(cycloalkyl), wherein cycloalkyl and alkyl are defined above, including —O—CH₂-cyclopropyl, —O—(CH₂)₂-cyclopropyl, —O—(CH₂)₃-cyclopropyl, —O—(CH₂)₄-cyclopropyl, O—CH₂-cyclobutyl, O—CH₂-cyclopentyl, O—CH₂-cyclohexyl, O—CH₂-cycloheptyl, and the like.

“Aminoalkoxy” means —O-(alkyl)-NH₂, wherein alkyl is defined above, such as —O—CH₂—NH₂, —O—(CH₂)₂—NH₂, —O—(CH₂)₃—NH₂, —O—(CH₂)₄—NH₂, —O—(CH₂)₅—NH₂, and the like.

“Mono-alkylamino” means —NH(alkyl), wherein alkyl is defined above, such as —NHCH₃, —NHCH₂CH₃, —NH(CH₂)₂CH₃, —NH(CH₂)₃CH₃, —NH(CH₂)₄CH₃, —NH(CH₂)₅CH₃, and the like.

“Di-alkylamino” means -N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃), and the like. “Mono-alkylaminoalkoxy” means —O-(alkyl)-NH(alkyl), wherein each alkyl is independently an alkyl group defined above, including —O—(CH₂)—NHCH₃, —O—(CH₂)—NHCH₂CH₃, —O—(CH₂)—NH(CH₂)₂CH₃, —O—(CH₂)—NH(CH₂)₃CH₃, —O—(CH₂)—NH(CH₂)₄CH₃, —O—(CH₂)—NH(CH₂)₅CH₃, —O—(CH₂)₂—NHCH₃, and the like.

“Di-alkylaminoalkoxy” means —O-(alkyl)-N(alkyl)(alkyl), wherein each alkyl is independently an alkyl group defined above, including —O—(CH₂)—N(CH₃)₂, —O—(CH₂)—N(CH₂CH₃)₂, —O—(CH₂)—N((CH₂)₂CH₃)₂, —O—(CH₂)—N(CH₃)(CH₂CH₃), and the like.

“Arylamino” means —NH(aryl), wherein aryl is defined above, including —NH(phenyl), —NH(tolyl), —NH(anthracenyl), —NH(fluorenyl), —NH(indenyl), —NH(azulenyl), —NH(pyridinyl), —NH(naphthyl), and the like.

“Arylalkylamino” means —NH-(alkyl)-(aryl), wherein alkyl and aryl are defined above, including —NH—CH₂-(phenyl), —NH—CH₂-(tolyl), —NH—CH₂-(anthracenyl), —NH—CH₂-(fluorenyl), —NH—CH₂-(indenyl), —NH—CH₂-(azulenyl), —NH—CH₂-(pyridinyl), —NH—CH₂-(naphthyl), —NH—(CH₂)₂-(phenyl) and the like.

“Alkylamino” means mono-alkylamino or di-alkylamino as defined above, such as —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃) and —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃) and the like.

“Cycloalkylamino” means —NH-(cycloalkyl), wherein cycloalkyl is as defined above, including —NH-cyclopropyl, —NH-cyclobutyl, —NH-cyclopentyl, —NH-cyclohexyl, —NH-cycloheptyl, and the like.

“Carboxyl” and “carboxy” mean —COOH.

“Cycloalkylalkylamino” means —NH-(alkyl)-(cycloalkyl), wherein alkyl and cycloalkyl are defined above, including —NH—CH₂-cyclopropyl, —NH—CH₂-cyclobutyl, —NH—CH₂-cyclopentyl, —NH—CH₂-cyclohexyl, —NH—CH₂-cycloheptyl, —NH—(CH₂)₂-cyclopropyl and the like.

“Aminoalkyl” means -(alkyl)-NH₂, wherein alkyl is defined above, including CH₂—NH₂, —(CH₂)₂—NH₂, —(CH₂)₃—NH₂, —(CH₂)₄—NH₂, —(CH₂)₅—NH₂ and the like.

“Mono-alkylaminoalkyl” means -(alkyl)-NH(alkyl), wherein each alkyl is independently an alkyl group defined above, including —CH₂—NH—CH₃, —CH₂—NHCH₂CH₃, —CH₂—NH(CH₂)₂CH₃, —CH₂—NH(CH₂)₃CH₃, —CH₂—NH(CH₂)₄CH₃, —CH₂—NH(CH₂)₅CH₃, —(CH₂)₂—NH—CH₃, and the like.

“Di-alkylaminoalkyl” means -(alkyl)-N(alkyl)(alkyl), wherein each alkyl is independently an alkyl group defined above, including —CH₂—N(CH₃)₂, —CH₂—N(CH₂CH₃)₂, —CH₂—N((CH₂)₂CH₃)₂, —CH₂—N(CH₃)(CH₂CH₃), —(CH₂)₂—N(CH₃)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, oxetanyl, azepinyl, piperazinyl, morpholinyl, dioxanyl, thietanyl and oxazolyl.

“Heteroarylalkyl” means -(alkyl)-(heteroaryl), wherein alkyl and heteroaryl are defined above, including —CH₂-triazolyl, —CH₂-tetrazolyl, —CH₂-oxadiazolyl, —CH₂-pyridyl, —CH₂-furyl, —CH₂-benzofuranyl, —CH₂-thiophenyl, —CH₂-benzothiophenyl, —CH₂-quinolinyl, —CH₂-pyrrolyl, —CH₂indolyl, —CH-oxazolyl, —CH₂-benzoxazolyl, —CH₂-imidazolyl, —CH₂-benzimidazolyl, —CH₂-thiazolyl, —CH₂-benzothiazolyl, —CH₂-isoxazolyl, —CH₂-pyrazolyl, —CH₂-isothiazolyl, —CH₂-pyridazinyl, —CH₂-pyrimidinyl, —CH₂-pyrazinyl, —CH₂-triazinyl, —CH₂-cinnolinyl, —CH₂-phthalazinyl, —CH₂-quinazolinyl, —CH₂-pyrimidyl, —CH₂-oxetanyl, —CH₂-azepinyl, —CH₂-piperazinyl, —CH₂-morpholinyl, —CH₂-dioxanyl, —CH₂-thietanyl, —CH₂-oxazolyl, —(CH₂)₂-triazolyl, and the like.

“Heterocycle” means a 5- to 7-membered monocyclic, or 7- to 10-membered bicyclic, heterocyclic ring which is either saturated, unsaturated, and which contains from 1 to 4 heteroatoms independently selected from nitrogen, oxygen and sulfur, and wherein the nitrogen and sulfur heteroatoms can be optionally oxidized, and the nitrogen heteroatom can be optionally quaternized, including bicyclic rings in which any of the above heterocycles are fused to a benzene ring. The heterocycle can be attached via any heteroatom or carbon atom. Heterocycles include heteroaryls as defined above. Representative heterocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocycle fused to phenyl” means a heterocycle, wherein heterocycle is defined as above, that is attached to a phenyl ring at two adjacent carbon atoms of the phenyl ring.

“Heterocycloalkyl” means -(alkyl)-(heterocycle), wherein alkyl and heterocycle are defined above, including —CH₂-morpholinyl, —CH₂-pyrrolidinonyl, —CH₂-pyrrolidinyl, —CH₂-piperidinyl, —CH₂-hydantoinyl, —CH₂-valerolactamyl, —CH₂-oxiranyl, —CH₂-oxetanyl, —CH₂-tetrahydrofuranyl, —CH₂-tetrahydropyranyl, —CH₂-tetrahydropyridinyl, —CH₂-tetrahydroprimidinyl, —CH₂-tetrahydrothiophenyl, —CH₂-tetrahydrothiopyranyl, —CH₂-tetrahydropyrimidinyl, —CH₂-tetrahydrothiophenyl, —CH₂-tetrahydrothiopyranyl, and the like.

The term “substituted” as used herein means any of the above groups (i.e., aryl, arylalkyl, heterocycle and heterocycloalkyl) wherein at least one hydrogen atom of the moiety being substituted is replaced with a substituent. In one embodiment, each carbon atom of the group being substituted is substituted with no more that two substituents. In another embodiment, each carbon atom of the group being substituted is substituted with no more than one substituent. In the case of a keto substituent, two hydrogen atoms are replaced with an oxygen which is attached to the carbon via a double bond. Substituents include halogen, hydroxyl, alkyl, haloalkyl, mono- or di-substituted aminoalkyl, alkyloxyalkyl, aryl, arylalkyl, heterocycle, heterocycloalkyl, —NRₐR_{b}, —NRₐC(═O)R_{b}, —NRₐC(═O)NRₐR_{b}, NRₐC(═O)OR_{b}—NRₐSO₂R_{b}, —ORₐ, —C(═O)RₐC(═O)ORₐ—C(═O)NRₐR_{b}, —OC(═O)Rₐ, —OC(═O)ORₐ, —OC(═O)NRₐR_{b}, —NRₐSO₂R_{b}, or a radical of the formula —Y—Z—Rₐ where Y is alkanediyl, or a direct bond, Z is —O—, —S—, —N(R_{b})—, —C(═O)—, —C(═O)O—, —OC(═O)—, —N(R_{b})C(═O)—, —C(═O)N(R_{b})— or a direct bond, wherein Rₐ and R_{b} are the same or different and independently hydrogen, amino, alkyl, haloalkyl, aryl, arylalkyl, heterocycle, or heterocylealkyl, or wherein Rₐ and R_{b} taken together with the nitrogen atom to which they are attached form a heterocycle.

“Haloalkyl” means alkyl, wherein alkyl is defined as above, having one or more hydrogen atoms replaced with halogen, wherein halogen is as defined above, including —CF₃, —CHF₂, —CH₂F, —CBr₃, —CHBr₂, —CH₂Br, —CCl₃, —CHCl₂, —CH₂Cl, —CI₃, —CHI₂, —CH₂I, —CH₂—CF₃, —CH₂—CHF₂, —CH₂—CH₂F, —CH₂—CBr₃, —CH₂—CHBr₂, —CH₂—CH₂Br, —CH₂—CCl₃, —CH₂—CHCl₂, —CH₂—CH₂Cl, —CH₂—CI₃, —CH₂—CHI₂, —CH₂—CH₂I, and the like.

“Hydroxyalkyl” means alkyl, wherein alkyl is as defined above, having one or more hydrogen atoms replaced with hydroxy, including —CH₂OH, —CH₂CH₂OH, —(CH₂)₂CH₂OH, —(CH₂)₃CH₂OH, —(CH₂)₄CH₂OH, —(CH₂)₅CH₂OH, —CH(OH)—CH₃, —CH₂CH(OH)CH₃, and the like.

“Hydroxy” means —OH.

“Sulfonyl” means —SO₃H.

“Sulfonylalkyl” means —SO₂-(alkyl), wherein alkyl is defined above, including —SO₂—CH₃, —SO₂—CH₂CH₃, —SO₂—(CH₂)₂CH₃, —SO₂—(CH₂)₃CH₃, —SO₂—(CH₂)₄CH₃, —SO₂—(CH₂)₅CH₃, and the like.

“Sulfinylalkyl” means —SO-(alkyl), wherein alkyl is defined above, including —SO—CH₃, —SO—CH₂CH₃, —SO—(CH₂)₂CH₃, —SO—(CH₂)₃CH₃, —SO—(CH₂)₄CH₃, —SO—(CH₂)₅CH₃, and the like.

“Sulfonamidoalkyl” means —NHSO₂-(alkyl), wherein aklyl is defined above, including —NHSO₂—CH₃, —NHSO₂—CH₂CH₃, —NHSO₂—(CH₂)₂CH₃, —NHSO₂—(CH₂)₃CH₃, —NHSO₂—(CH₂)₄CH₃, —NHSO₂—(CH₂)₅CH₃, and the like.

“Thioalkyl” means —S-(alkyl), wherein alkyl is defined above, including —S—CH₃, —S—CH₂CH₃, —S—(CH₂)₂CH₃, —S—(CH₂)₃CH₃, —S—(CH₂)₄CH₃, —S—(CH₂)₅CH₃, and the like.

As used herein, the term “JNK Inhibitor” means a compound capable of inhibiting the activity of JNK in vitro or in vivo. The JNK Inhibitor can be in the form of a pharmaceutically acceptable salt, free base, solvate, hydrate, stereoisomer, clathrate or prodrug thereof. Such inhibitory activity can be determined by an assay or animal model well-known in the art including those set forth in Section 5. In one embodiment, the JNK Inhibitor is a compound of structure (I)-(III).

“JNK” means a protein or an isoform thereof expressed by a JNK 1, JNK 2, or JNK 3 gene (Gupta, S., Barrett, T., Whitmarsh, A. J., Cavanagh, J., Sluss, H. K., Derijard, B. and Davis, R. J. The EMBO J. 15:2760-2770 (1996)).

As used herein, the terms “asbestos-related disease, disorder or syndrome,” “disease or disorder associated with asbestos exposure,” and “disease or disorder associated with asbestos poisoning” mean any disease, disorder, syndrome or abnormality associated with, or related to, exposure to asbestos or poisoning by asbestos. The terms encompass benign and malignant diseases or disorders, and include, but are not limited to, mesothelioma, fibrosis, asbestosis, malignant pleural effusion, benign exudative effusion, pleural plaques, pleural calcification, diffuse pleural thickening, rounded atelectasis, fibrotic masses, and lung cancer. In a specific embodiment, the terms do not encompass lung cancer and in another embodiment do not include fibrosis.

As used herein, the phrase “an effective amount” when used in connection with a JNK Inhibitor means an amount of the JNK Inhibitor that is useful for treating, preventing, and/or managing an asbestos-related disease or disorder.

As used herein, the phrase “an effective amount” when used in connection with another active agent means an amount of the other active agent that is useful for treating, preventing, and/or managing an asbestos-related disease or disorder when administered while the JNK Inhibitor exerts its therapeutic or prophylactic activity.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the JNK Inhibitor include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “clathrate” means a JNK Inhibitor, or a salt thereof, in the form of a crystal lattice that contains spaces (e.g., channels) that have a guest molecule (e.g., a solvent or water) trapped within.

As used herein and unless otherwise indicated, the term “hydrate” means a JNK inhibitor, or a salt thereof, that further includes a stoichiometric or non-stoichiometric amount of water bound by non-covalent intermolecular forces.

As used herein and unless otherwise indicated, the term “polymorph” means a particular crystalline arrangement of the JNK Inhibitor. Polymorphs can be obtained through the use of different work-up conditions and/or solvents. In particular, polymorphs can be prepared by recrystallization of a JNK Inhibitor in a particular solvent.

As used herein and unless otherwise indicated, the term “prodrug” means a JNK Inhibitor derivative that can hydrolyze, oxidize, or otherwise react under biological conditions (in vitro or in vivo) to provide an active compound, particularly a JNK Inhibitor. Examples of prodrugs include, but are not limited to, derivatives and metabolites of a JNK Inhibitor that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound is substantially free of other stereoisomers of that compound. For example, a stereomerically pure compound having one chiral center will be substantially free of the opposite enantiomer of the compound. A stereomerically pure a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

### 4. DETAILED DESCRIPTION OF THE INVENTION

A first embodiment of the invention encompasses methods of treating, preventing and/or managing an asbestos-related disease or disorder, which comprises administering to a patient in need thereof an effective amount of a JNK Inhibitor.

Another embodiment of the invention encompasses a pharmaceutical composition suitable for treatment, prevention and/or management of asbestos-related diseases or disorders comprising an effective amount of a JNK Inhibitor.

Also encompassed by the invention are single unit dosage forms suitable for use in treating, preventing and/or managing asbestos-related diseases or disorders comprising an effective amount of a JNK Inhibitor, and an optional vehicle, carrier or excipient.

Another embodiment of the invention encompasses a kit suitable for use in treating, preventing and/or managing asbestos-related diseases or disorders comprising: a pharmaceutical composition comprising an effective amount of a JNK Inhibitor. The invention further encompasses kits comprising single unit dosage forms.

Without being limited by theory, it is believed that a JNK Inhibitor can act in complementary or synergistic ways with certain second active agents in the treatment, prevention and/or management of asbestos-related diseases or disorders. Therefore, one embodiment of the invention encompasses a method of treating, preventing and/or managing an asbestos-related disease or disorder, which comprises administering to a patient in need thereof an effective amount of a JNK Inhibitor, and an effective amount of a second active agent.

Examples of second active agents include, but are not limited to, conventional therapeutics used to treat or prevent mesothelioma such as anti-cancer agents, antibiotics, anti-inflammatory agents, steroids, cytokines, immunomodulatory agents, immunosuppressive agents, and other therapeutics drug capable of relieving or alleviating a symptom of asbestos-related diseases or disorders which can be found, for example, in the Physician's Desk Reference, 2003.

It is further believed that a JNK Inhibitor can reduce or eliminate adverse effects associated with the administration of conventional therapeutic agents used to treat asbestos-related diseases or disorders, thereby allowing the administration of larger amounts of those conventional agents to patients and/or increasing patient compliance. Consequently, another embodiment of the invention encompasses a method of reversing, reducing or avoiding an adverse effect associated with the administration of a second active agent in a patient suffering from an asbestos-related disease or disorder, which comprises administering to a patient in need thereof an effective amount of a JNK Inhibitor.

The invention also encompasses pharmaceutical compositions, single unit dosage forms, and kits which comprise an effective amount of a JNK Inhibitor and an effective amount of a second active agent.

As discussed elsewhere herein, symptoms of asbestos-related diseases or disorders may be treated with chemotherapy, surgery, radiation therapy, photodynamic therapy, immunotherapy, and/or gene therapy. Without being limited by theory, it is believed that the combined use of such conventional therapies and a JNK Inhibitor can provide a uniquely effective treatment of asbestos-related diseases or disorders. Therefore, this invention encompasses a method of treating, preventing and/or managing asbestos-related diseases or disorders, which comprises administering to a patient (e.g., a human) an effective amount of a JNK Inhibitor, before, during, or after chemotherapy, surgery, radiation therapy, photodynamic therapy, immunotherapy, gene therapy and/or other conventional, non-drug based therapies.

### 4.1 Illustrative JNK Inhibitors

As mentioned above, the present invention is directed to methods useful for treating, preventing and/or managing asbestos-related diseases or disorders, comprising administering an effective amount of a JNK Inhibitor to a patient in need thereof. Illustrative JNK Inhibitors are set forth below.

In one embodiment, the JNK Inhibitor has the following structure (I):

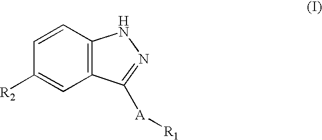

wherein:

A is a direct bond, —(CH₂)ₐ—, —(CH₂)_{b}CH═CH(CH₂)_{c}—, or —(CH₂)_{b}C≡C(CH₂)_{c}—;

R₁ is aryl, heteroaryl or heterocycle fused to phenyl, each being optionally substituted with one to four substituents independently selected from R₃;

R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;

a is 1, 2, 3, 4, 5 or 6;

b and c are the same or different and at each occurrence independently selected from 0, 1, 2, 3 or 4;

d is at each occurrence 0, 1 or 2;

R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;

R₄ is alkyl, aryl, arylalkyl, heterocycle or heterocycloalkyl, each being optionally substituted with one to four substituents independently selected from R₃, or R₄ is halogen or hydroxy;

R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and

R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

In one embodiment, -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3 and wherein R₈ and R₉ are defined above.

In another embodiment, R₂ is —R₄, —(CH₂)_{b}C(═O)R₅, —(CH₂)_{b}C(═O)OR₅, —(CH₂)_{b}C(═O)NR₅R₆, —CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆, and b is an integer ranging from 0-4.

In another embodiment, R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In another embodiment, R₂ is 3-triazolyl or 5-tetrazolyl.

In another embodiment:

(a) -A-R₁ is phenyl, optionally substituted with one to four substituents independently selected from halogen, alkoxy, —NR₈C(═O)R₉, —C(═O)NR₈R₉,

and —O(CH₂)_{b}NR₈R₉, wherein b is 2 or 3; and

(b) R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In another embodiment:

(a) -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3; and

(b) R₂ is 3-triazolyl or 5-tetrazolyl.

In another embodiment, R₂ is R₄, and R₄ is 3-triazolyl, optionally substituted at its 5-position with:

(a) a C₁-C₄ straight or branched chain alkyl group optionally substituted with a hydroxyl, methylamino, dimethylamino or 1-pyrrolidinyl group; or

(b) a 2-pyrrolidinyl group.

In another embodiment, R₂ is R₄, and R₄ is 3-triazolyl, optionally substituted at its 5-position with: methyl, n-propyl, isopropyl, 1-hydroxyethyl, 3-hydroxypropyl, methylaminomethyl, dimethylaminomethyl, 1-(dimethylamino)ethyl, 1-pyrrolidinylmethyl or 2-pyrrolidinyl.

In another embodiment, the compounds of structure (I) have structure (IA) when A is a direct bond, or have structure (IB) when A is —(CH₂)ₐ—:

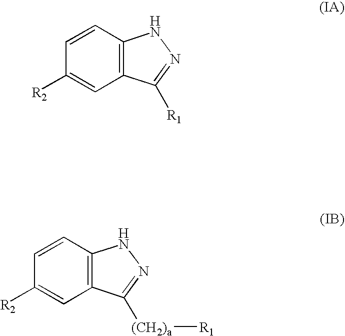

In other embodiments, the compounds of structure (I) have structure (IC) when A is a —CH₂)_{b}CH═CH(CH₂)_{c}—, and have structure (ID) when A is —(CH₂)_{b}C≡C(CH₂)_{c}—:

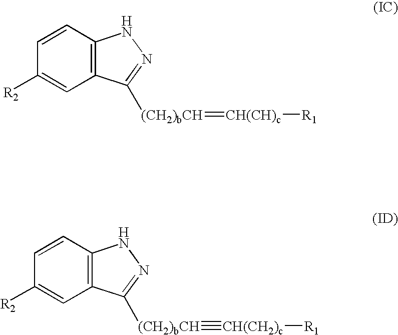

In further embodiments of this invention, R₁ of structure (I) is aryl or substituted aryl, such as phenyl or substituted phenyl as represented by the following structure (IE):

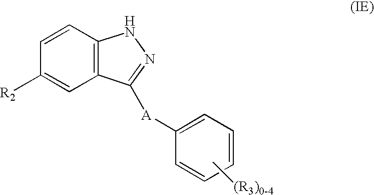

In another embodiment, R₂ of structure (I) is —(CH₂)_{b}NR₄(C═O)R₅. In one aspect of this embodiment, b=0 and the compounds have the following structure (IF):

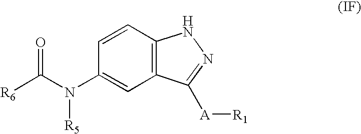

Representative R₂ groups of the compounds of structure (I) include alkyl (such as methyl and ethyl), halo (such as chloro and fluoro), haloalkyl (such as trifluoromethyl), hydroxy, alkoxy (such as methoxy and ethoxy), amino, arylalkyloxy (such as benzyloxy), mono- or di-alkylamine (such as —NHCH₃, —N(CH₃)₂ and —NHCH₂CH₃), —NHC(═O)R₄ wherein R₆ is a substituted or unsubstituted phenyl or heteroaryl (such as phenyl or heteroaryl substituted with hydroxy, carboxy, amino, ester, alkoxy, alkyl, aryl, haloalkyl, halo, —CONH₂ and —CONH alkyl), —NH(heteroarylalkyl) (such as —NHCH₂(3-pyridyl), —NHCH₂(4-pyridyl), heteroaryl (such as pyrazolo, triazolo and tetrazolo), —C(═O)NHR₆ wherein R₆ is hydrogen, alkyl, or as defined above (such as —C(═O)NH₂, —C(═O)NHCH₃, —C(═O)NH(H-carboxyphenyl), —C(═O)N(CH₃)₂), arylalkenyl (such as phenylvinyl, 3-nitrophenylvinyl, 4-carboxyphenylvinyl), heteroarylalkenyl (such as 2-pyridylvinyl, 4-pyridylvinyl).

Representative R₃ groups of the compounds of structure (I) include halogen (such as chloro and fluoro), alkyl (such as methyl, ethyl and isopropyl), haloalkyl (such as trifluoromethyl), hydroxy, alkoxy (such as methoxy, ethoxy, n-propyloxy and isobutyloxy), amino, mono- or di-alkylamino (such as dimethylamine), aryl (such as phenyl), carboxy, nitro, cyano, sulfinylalkyl (such as methylsulfinyl), sulfonylalkyl (such as methylsulfonyl), sulfonamidoalkyl (such as —NHSO₂CH₃), —NR₈C(═O)(CH₂)_{b}OR₉ (such as NHC(═O)CH₂OCH₃), NHC(═O)R₉(such as —NHC(═O)CH₃, —NHC(═O)CH₂C₆H₅, —NHC(═O)(2-furanyl)), and —O(CH₂)_{b}NR₈R₉ (such as —O(CH₂)₂N(CH₃)₂).

The compounds of structure (I) can be made using organic synthesis techniques known to those skilled in the art, as well as by the methods described in International Publication No. WO 02/10137 (particularly in Examples 1-430, at page 35, line 1 to page 396, line 12), published Feb. 7, 2002, which is incorporated herein by reference in its entirety. Further, specific examples of these compounds are found in this publication.

Illustrative examples of JNK Inhibitors of structure (I) are: 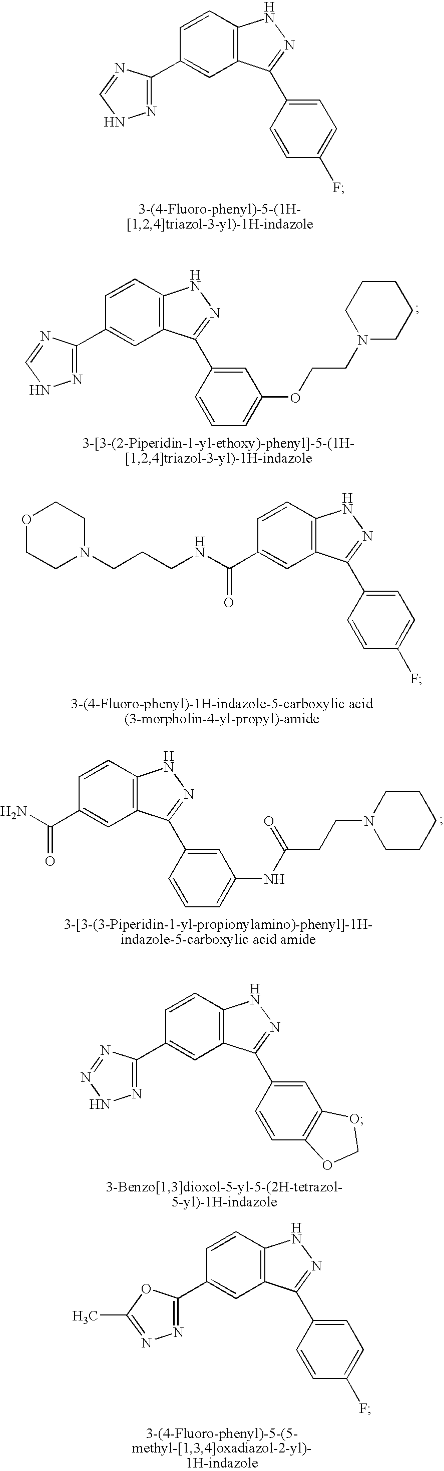 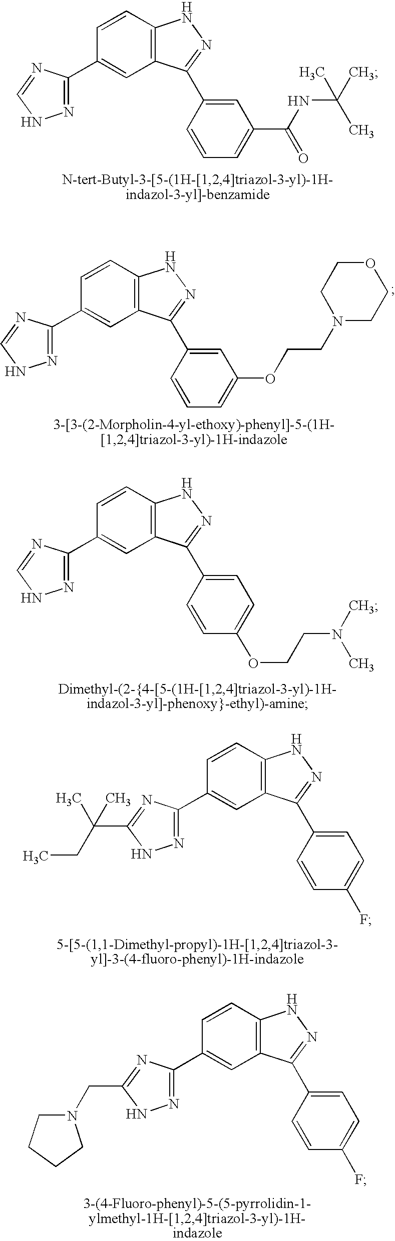 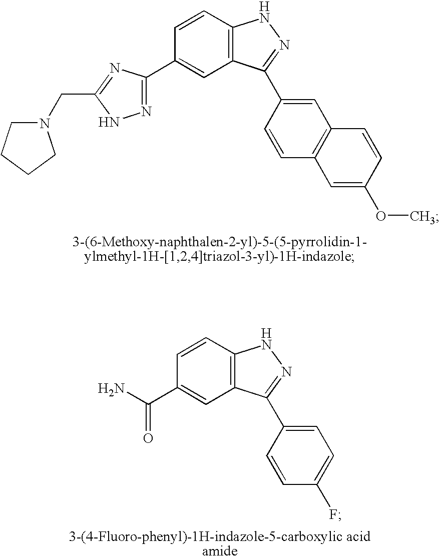
and pharmaceutically acceptable salts thereof.

In another embodiment, the JNK Inhibitor has the following structure (II):

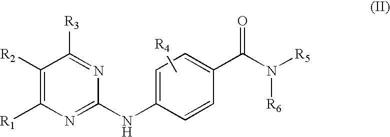

wherein:

R₁ is aryl or heteroaryl optionally substituted with one to four substituents independently selected from R₇;

R₂ is hydrogen;

R₃ is hydrogen or lower alkyl;

R₄ represents one to four optional substituents, wherein each substituent is the same or different and independently selected from halogen, hydroxy, lower alkyl and lower alkoxy;

R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;

or R₅ and R₆ taken together with the nitrogen atom to which they are attached to form a heterocycle or substituted heterocycle;

R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, substituted heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;

R₈, R₉, R₁₀ and R₁₁, are the same or different and at each occurrence independently hydrogen, alkyl, aryl, arylalkyl, heterocycle, heterocycloalkyl;

or R₈ and R₉ taken together with the atom or atoms to which they are attached to form a heterocycle;

a and b are the same or different and at each occurrence independently selected from 0, 1, 2, 3 or 4; and

c is at each occurrence 0, 1 or 2.

In one embodiment, R₁ is a substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl or quinazolinyl.

In another embodiment R₁ is substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, preferably phenyl. When R₁ is a substituted aryl, the substituents are defined below. In one embodiment, when substituted, R₁ is substituted with a halogen, —SO₂R₈ or —SO₂R₈R₉.

In another embodiment, R₅ and R₆, taken together with the nitrogen atom to which they are attached form a substituted or unsubstituted nitrogen-containing non-aromatic heterocycle, in one embodiment, piperazinyl, piperidinyl or morpholinyl.

When R₅ and R₆, taken together with the nitrogen atom to which they are attached form substituted piperazinyl, piperazinyl or morpholinyl, the piperazinyl, piperadinyl or morpholinyl is substituted with one or more substituents defined below. In one embodiment, when substituted, the substituent is alkyl, amino, alkylamino, alkoxyalkyl, acyl, pyrrolidinyl or piperidinyl.

In one embodiment, R₃ is hydrogen and R₄ is not present, and the JNK Inhibitor has the following structure (IIA): 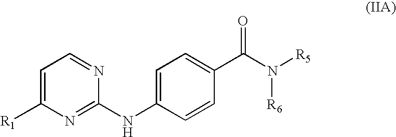
and pharmaceutically acceptable salts thereof.

In a more specific embodiment, R₁ is phenyl optionally substituted with R₇, and having the following structure (IIB): 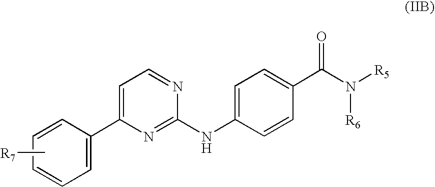
and pharmaceutically acceptable salts thereof.

In still a further embodiment, R₇ is at the para position of the phenyl group relative to the pyrimidine, as represented by the following structure (IIC): 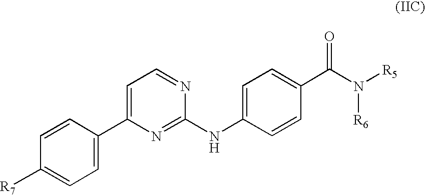
and pharmaceutically acceptable salts thereof.

The JNK Inhibitors of structure (II) can be made using organic synthesis techniques known to those skilled in the art, as well as by the methods described in International Publication No. WO 02/46170 (particularly Examples 1-27 at page 23, line 5 to page 183, line 25), published Jun. 13, 2002, which is hereby incorporated by reference in its entirety. Further, specific examples of these compounds are found in the publication.

Illustrative examples of JNK Inhibitors of structure (II) are: 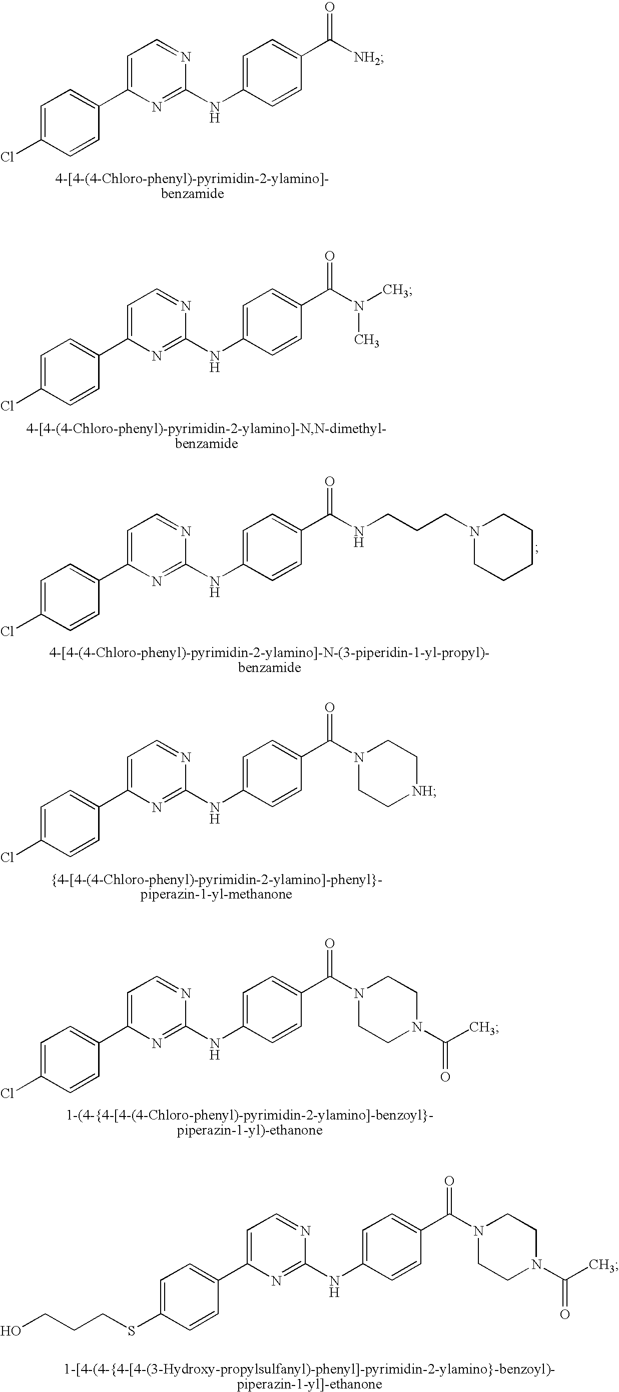 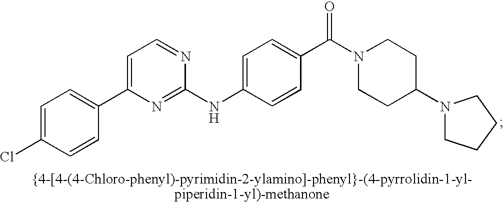
and pharmaceutically acceptable salts thereof.

In another embodiment, the JNK Inhibitor has the following structure (III):

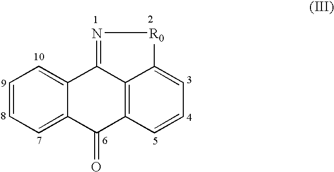

wherein R₀ is —O—, —S—, —S(O)—, —S(O)₂—, NH or —CH₂—;

the compound of structure (III) being: (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

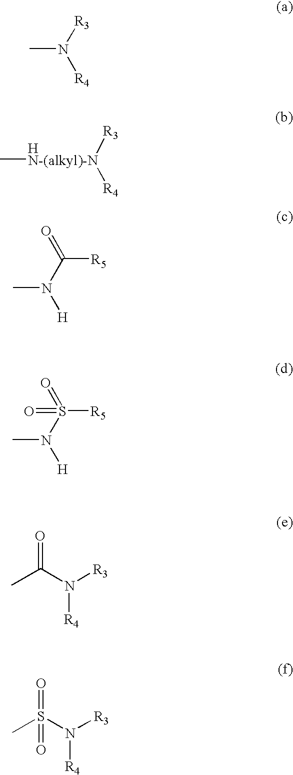

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

In another embodiment, the JNK Inhibitor has the following structure (IIIA):

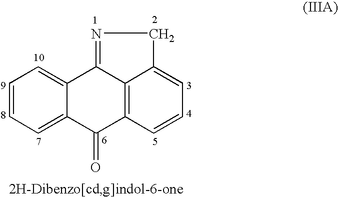

being: (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position;

wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

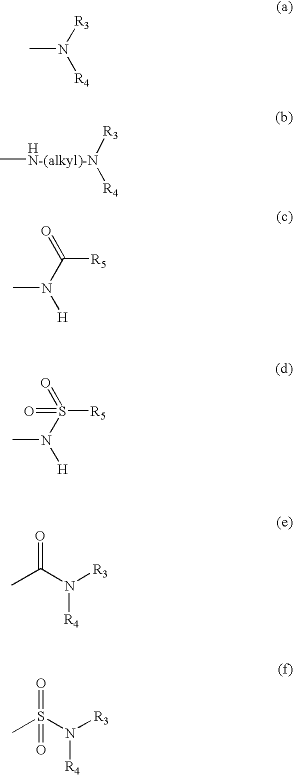

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIA) is that wherein the first or second substituent is present at the 5, 7, or 9 position. In one embodiment, the first or second substituent is present at the 5 or 7 position.

A second subclass of compounds of structure (IIIA) is that wherein the first or second substituent is present at the 5, 7, or 9 position;

the first or second substituent is independently alkoxy, aryloxy, aminoalkyl, mono-alkylaminoalkyl, di-alkylaminoalkyl, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK Inhibitor has the following structure (IIIB):

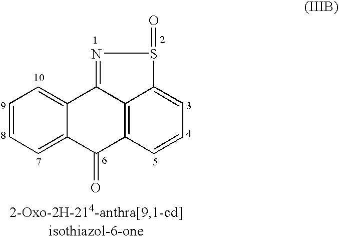

being (i) unsubstituted, (ii) monosubstituted and having a first substituent, or (ii) disubstituted and having a first substituent and a second substituent;

the first or second substituent, when present, is at the 3, 4, 5, 7, 8, 9, or 10 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b) (c), (d), (e), or (f):

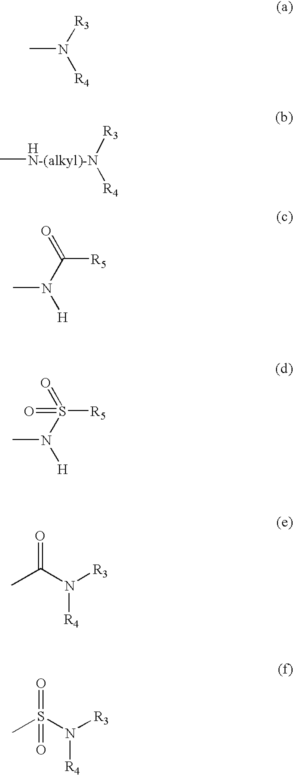

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIB) is that wherein the first or second substituent is present at the 5, 7, or 9 position. In one embodiment, the first or second substituent is present at the 5 or 7 position.

A second subclass of the compounds of structure (IIIB) is that wherein the first or second substituent is independently alkoxy, aryloxy, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK Inhibitor has the following structure (IIIC):

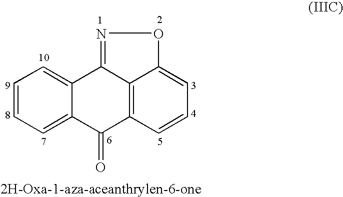

being (i) monosubstituted and having a first substituent or (ii) disubstituted and haying a first substituent and a second substituent;

the first or second substituent, when present, is at the 3, 4, 5, 7, 8, 9, or 10 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c) (d), (e), or (f):

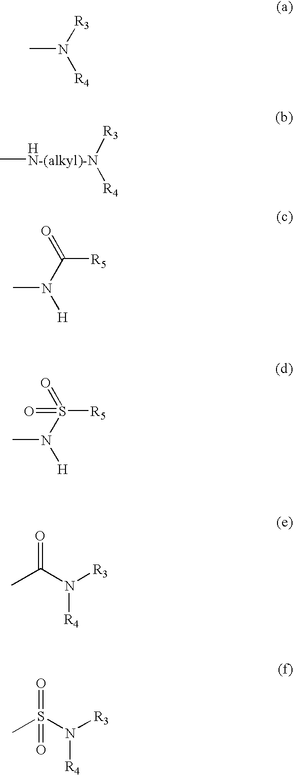

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIC) is that wherein the first or second substituent is present at the 5, 7, or 9 position. In one embodiment, the first or second substituent is present at the 5 or 7 position.

A second subclass of the compounds of structure (IIIC) is that wherein the first or second substituent is independently alkoxy, aryloxy, aminoalkyl, mono-alkylaminoalkyl, di-alkylaminoalkyl, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK Inhibitor has the following structure (IIID):

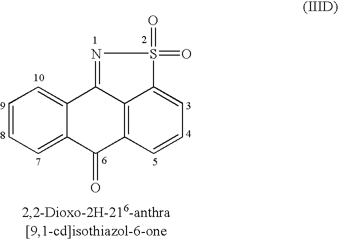

being (i) monosubstituted and having a first substituent present at the 5, 7, or 9 position, (ii) disubstituted and having a first substituent present at the 5 position and a second substituent present at the 7 position, (iii) disubstituted and having a first substituent present at the 5 position and a second substituent present at the 9 position, or (iv) disubstituted and having a first substituent present at the 7 position and a second substituent present at the 9 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

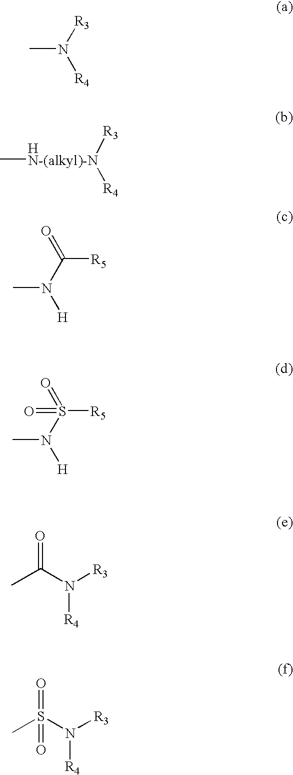

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIID) is that wherein the first or second substituent is present at the 5 or 7 position.

A second subclass of the compounds of structure (IIID) is that wherein the first or second substituent is independently alkyl, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (c), (d), (e), or (f).

Another subclass of the compounds of structure (IIID) is that wherein the first and second substituent are independently alkoxy, aryloxy, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, alkoxycarbonyl, or cycloalkylalkyl.

In another embodiment, the JNK Inhibitor has the following structure (IIIE):

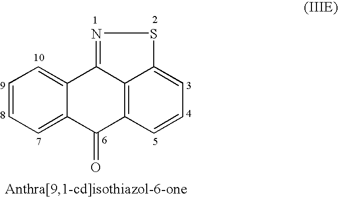

being (i) monosubstituted and having a first substituent present at the 5, 7, or 9 position, (ii) disubstituted and having a first substituent present at the 5 position and a second substituent present at the 9 position, (iii) disubstituted and having a first substituent present at the 7 position and a second substituent present at the 9 position, or (iv) disubstituted and having a first substituent present at the 5 position and a second substituent present at the 7 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

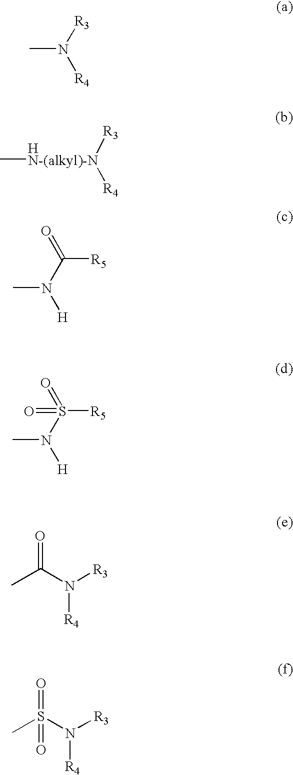

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIE) is that wherein the first or second substituent is present at the 5 or 7 position.

A second subclass of the compounds of structure (IIIE) is that wherein the compound of structure (IIIE) is disubstituted and at least one of the substituents is a group represented by the structure (d) or (f).

Another subclass of the compounds of structure (IIIE) is that wherein the compounds are monosubstituted. Yet another subclass of compounds is that wherein the compounds are monosubstituted at the 5 or 7 position with a group represented by the structure (e) or (f).

In another embodiment, the JNK Inhibitor has the following structure (IIIF):

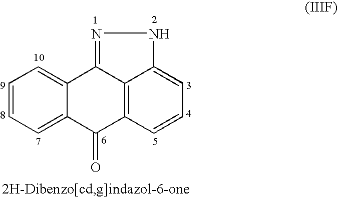

being (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position;

wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

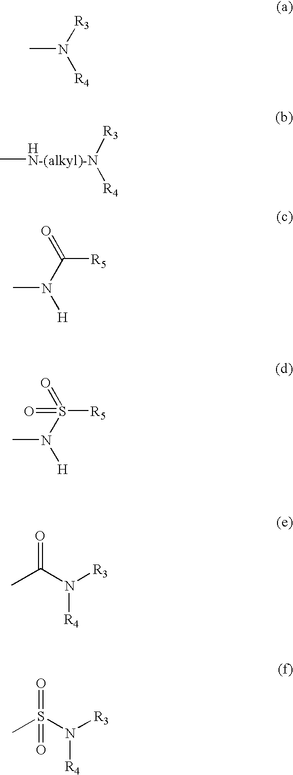

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

In one embodiment, the compound of structure (IIIF), or a pharmaceutically acceptable salt thereof is unsubstituted at the 3, 4, 5, 7, 8, 9, or 10 position.

The JNK Inhibitors of structure (III) can be made using organic synthesis techniques known to those skilled in the art, as well as by the methods described in International Publication No. WO 01/12609 (particularly Examples 1-7 at page 24, line 6 to page 49, line 16), published Feb. 22, 2001, as well as International Publication No. WO 02/066450 (particularly compounds AA-HG at pages 59-108), published Aug. 29, 2002, each of which is hereby incorporated by reference in its entirety. Further, specific examples of these compounds can be found in the publications.

Illustrative examples of JNK Inhibitors of structure (III) are: 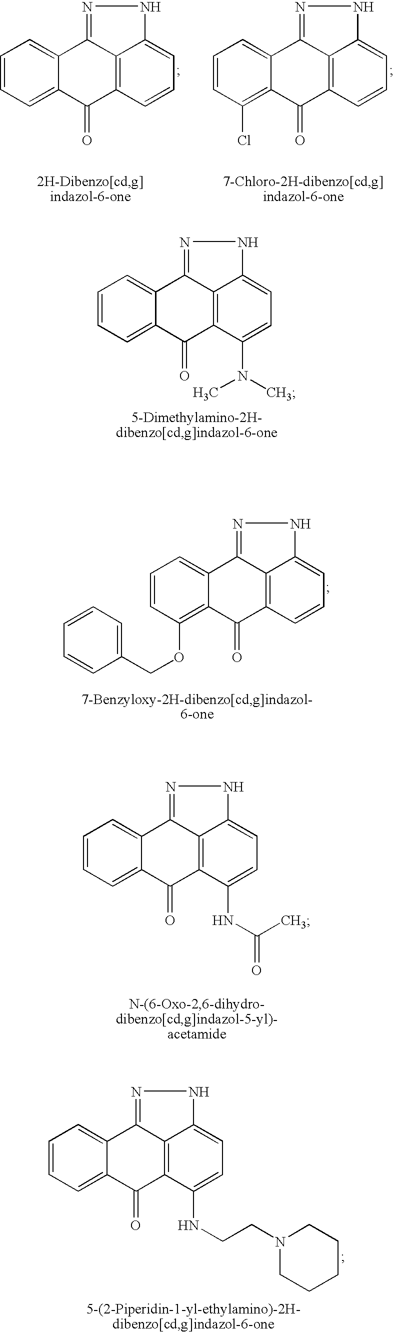 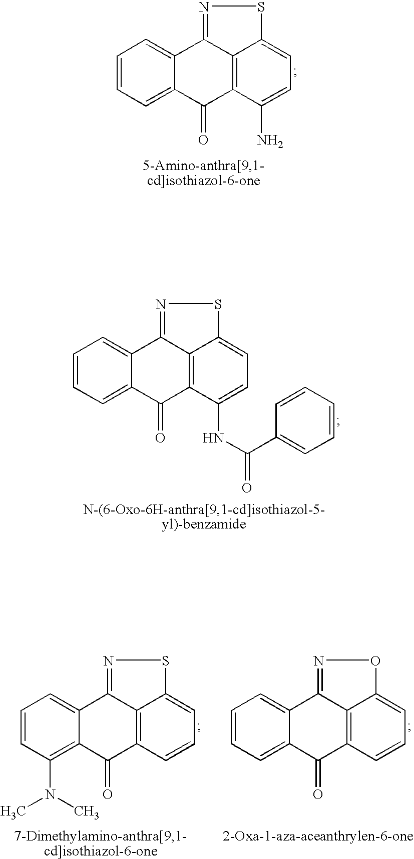
and pharmaceutically acceptable salts thereof.

Other JNK Inhibitors that are useful in the present methods include, but are not limited to, those disclosed in International Publication No. WO 00/39101, (particularly at page 2, line 10 to page 6, line 12); International Publication No. WO 01/14375 (particularly at page 2, line 4 to page 4, line 4); International Publication No. WO 00/56738 (particularly at page 3, line 25 to page 6, line 13); International Publication No. WO 01/27089 (particularly at page 3, line 7 to page 5, line 29); International Publication No. WO 00/12468 (particularly at page 2, line 10 to page 4, line 14); European Patent Publication 1 110 957 (particularly at page 19, line 52 to page 21, line 9); International Publication No. WO 00/75118 (particularly at page 8, line 10 to page 11, line 26); International Publication No. WO 01/12621 (particularly at page 8, line 10 to page 10, line 7); International Publication No. WO 00/64872 (particularly at page 9, line 1 to page, 106, line 2); International Publication No. WO 01/23378 (particularly at page 90, line 1 to page 91, line 11); International Publication No. WO 02/16359 (particularly at page 163, line 1 to page 164, line 25); U.S. Pat. No. 6,288,089 (particularly at column 22, line 25 to column 25, line 35); U.S. Pat. No. 6,307,056 (particularly at column 63, line 29 to column 66, line 12); International Publication No. WO 00/35921 (particularly at page 23, line 5 to page 26, line 14); International Publication No. WO 01/91749 (particularly at page 29, lines 1-22); International Publication No. WO 01/56993 (particularly in at page 43 to page 45); and International Publication No. WO 01/58448 (particularly in at page 39), each of which is incorporated by reference herein in its entirety.

Pharmaceutical compositions including dosage forms of the invention, which comprise an effective amount of a JNK Inhibitor can be used in the methods of the invention.

### 4.2 Methods of Use

Methods of this invention encompass methods of treating, preventing and/or managing various types of asbestos-related diseases or disorders. As used herein, unless otherwise specified, the term “treating” refers to the administration of an effective amount of a JNK Inhibitor after the onset of symptoms of asbestos-related diseases or disorders, whereas “preventing” refers to the administration prior to the onset of symptoms, particularly to patients at risk of mesothelioma or other asbestos-related disorders. The term “preventing” further includes the inhibiting or averting a symptom of the particular disease or disorder. Symptoms of asbestos-related diseases or disorders include, but are not limited to, dyspnea, obliteration of the diaphragm, radiolucent sheet-like encasement of the pleura, pleural effusion, pleural thickening, decreased size of the chest, chest discomfort, chest pain, easy fatigability, fever, sweats and weight loss. Examples of patients at risk of asbestos-related diseases or disorders include, but are not limited to, those who have been exposed to asbestos in the workplace and their family members who have been exposed to asbestos embedded in the worker's clothing. Patients having familial history of asbestos-related diseases or disorders are also preferred candidates for preventive regimens.

As used herein and unless otherwise indicated, the term “managing asbestos-related diseases or disorders” encompasses preventing the recurrence of the diseases or disorders in a patient who had suffered from the diseases or disorders, and/or lengthening the time that a patient who had suffered from those remains in remission.

In one embodiment, methods encompassed by this invention comprise administering an effective amount of a JNK Inhibitor to a patient (e.g., a human) suffering, or likely to suffer, from asbestos-related diseases or disorders.

Without being limited by theory, it is believed that a JNK Inhibitor can be prophylactically administered to prevent people who have been previously exposed to asbestos from developing asbestos-related diseases or disorders. This prophylactic method can actually prevent asbestos-related diseases or disorders from developing in the first place. Therefore, the invention further encompasses a method for preventing asbestos-related diseases or disorders in people who are at risk of asbestos-related diseases or disorders, comprising administering an effective amount of a JNK Inhibitor to a patient in need thereof.

Without being limited by theory, it is also believed that a JNK Inhibitor can inhibit spread of asbestos-related diseases or disorders after diagnosis, because the compounds can affect the production of cytokines (e.g., TNF-α).

The invention encompasses methods for treating, preventing and/or managing asbestos-related diseases or disorders in patients with various stages and specific types of the diseases, including, but not limited to, malignant mesothelioma, asbestosis, malignant pleural effusion, benign pleural effusion, pleural plaque, pleural calcification, diffuse pleural thickening, round atelectasis, and bronchogenic carcinoma. It further encompasses methods of treating patients who have been previously treated for asbestos-related diseases or disorders but were not sufficiently responsive or were non-responsive, as well as those who have not previously been treated for the diseases or disorders. Because patients have heterogenous clinical manifestations and varying clinical outcomes, the treatment given to a patient may vary, depending on his/her prognosis. The skilled clinician will be able to readily determine without undue experimentation specific secondary agents and types of physical therapy that can be effectively used to treat an individual patient.

In one embodiment of the invention, a JNK Inhibitor is administered orally and daily in an amount of from about 1 mg to about 10,000 mg. More specifically, the daily dose is administered twice daily in equally divided doses. Specifically, a daily dose range can be from about 1 mg to about 5,000 mg per day, from about 10 mg to about 2,500 mg per day, from about 100 mg to about 800 mg per day, from about 100 mg to about 1,200 mg per day, or from about 25 mg to about 2,500 mg per day. In managing the patient, the therapy should be initiated at a lower dose, perhaps about 1 mg to about 2,500 mg, and increased if necessary up to about 200 mg to about 5,000 mg per day as either a single dose or divided doses, depending on the patient's global response.

### 4.2.1 Combination Therapy with A Second Active Agent

The invention further relates to methods for treating, preventing and/or managing an asbestos-related disease or disorder, comprising administering an effective amount of a JNK Inhibitor in combination with an effective amount of a second active agent, such as a prophylactic or therapeutic agent, to a patient in need thereof.

It is believed that certain combinations work synergistically in the treatment of asbestos-related diseases or disorders. A JNK Inhibitor can also work to alleviate adverse effects associated with certain second active agents, and some second active agents can be used to alleviate adverse effects associated with a JNK Inhibitor.

One or more second active agents can be used in the methods and compositions of the invention together with a JNK Inhibitor. Second active agents can be large molecules (e.g., proteins) or small molecules (e.g., synthetic inorganic, organometallic, or organic molecules).

Examples of large molecule active agents are biological molecules, such as naturally occurring or artificially made proteins. Particular proteins include, but are not limited to: cytokines such as GM-CSF, interleukins such as IL-2 (including recombinant IL-II (“rIL2”) and canarypox IL-2), IL-10, IL-12, and IL-18; and interferons, such as interferon alfa-2a, interferon alfa-2b, interferon alfa-n1, interferon alfa-n3, interferon beta-Ia, and interferon gamma-Ib.

In one embodiment of the invention, the large molecule active agent reduces, eliminates, or prevents an adverse effect associated with the administration of a JNK Inhibitor. Depending on the disease or disorder begin treated, adverse effects can include, but are not limited to, drowsiness, somnolence, nausea, emesis, gastrointestinal discomfort, diarrhea, and vasculitis.

Second active agents that are small molecules can also be used to alleviate adverse effects associated with the administration of a JNK Inhibitor. Like some large molecules, many are believed to be capable of providing a synergistic effect when administered with (e.g., before, after or simultaneously) a JNK Inhibitor. Examples of small molecule second active agents include, but are not limited to, anti-cancer agents, antibiotics, anti-inflammatory agents, IMiDs® and SelCIDs® (Celgene Corporation, New Jersey) (e.g., those disclosed in U.S. Pat. Nos. 6,075,041; 5,877,200; 5,698,579; 5,703,098; 6,429,221; 5,736,570; 5,658,940; 5,728,845; 5,728,844; 6,262,101; 6,020,358; 5,929,117; 6,326,388; 6,281,230; 5,635,517; 5,798,368; 6,395,754; 5,955,476; 6,403,613; 6,380,239; and 6,458,810, each of which is incorporated herein by reference) and steroids.

Examples of anti-cancer agents include, but are not limited to: acivicin; aclarubicin; acodazole hydrochloride; acronine; 4-(amino)-2-(2,6-dioxo(3-piperidyl))-isoindoline-1,3-dione (Actimid™); adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; 3-(4-amino-1-oxo-1,3-dihydro-isoindol-2-yl)-piperidine-2,6-dione (Revimid™); riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear poiyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific second active agents include, but are not limited to, anthracycline, platinum, alkylating agent, oblimersen (Genasense®), gemcitabine, cisplatinum, cyclophosphamide, temodar, carboplatin, procarbazine, gliadel, tamoxifen, methotrexate, taxotere, irinotecan, topotecan, temozolomide, capecitabine, cisplatin, thiotepa, fludarabine, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, bleomycin, hyaluronidase, mepacrine, thiotepa, tetracycline, thalidomide and mitomycin C.

In one embodiment, a JNK Inhibitor and a second active agent are administered to a patient, preferably a mammal, more preferably a human, in a sequence and within a time interval such that the JNK Inhibitor can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In one embodiment, the JNK Inhibitor and the second active agent exert their effect at times which overlap. Each second active agent can be administered separately, in any appropriate form and by any suitable route. In other embodiments, the JNK Inhibitor is administered before, concurrently or after administration of the second active agent.

In various embodiments, the JNK Inhibitor and the second active agent are administered less than about 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In other embodiments, the JNK Inhibitor and the second active agent are administered concurrently.

In other embodiments, the JNK Inhibitor and the second active agent are administered at about 2 to 4 days apart, at about 4 to 6 days apart, at about 1 week part, at about 1 to 2 weeks apart, or more than 2 weeks apart.

In certain embodiments, the JNK Inhibitor and optionally the second active agent are cyclically administered to a patient. Cycling therapy involves the administration of a first agent for a period of time, followed by the administration of a second agent and/or third agent for a period of time and repeating this sequential administration. Cycling therapy can reduce the development of resistance to one or more of the therapies, avoid or reduce the side effects of one of the therapies, and/or improve the efficacy of the treatment.

In certain embodiments, the JNK Inhibitor and optionally the second active agent are administered in a cycle of less than about 3 weeks, about once every two weeks, about once every 10 days or about once every week. One cycle can comprise the administration of a JNK Inhibitor and optionally the second active agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In yet other embodiments, the JNK Inhibitor is administered in metronomic dosing regimens, either by continuous infusion or frequent administration without extended rest periods. Such metronomic administration can involve dosing at constant intervals without rest periods. Typically the JNK Inhibitors, are used at lower doses. Such dosing regimens encompass the chronic daily administration of relatively low doses for extended periods of time. In preferred embodiments, the use of lower doses can minimize toxic side effects and eliminate rest periods. In certain embodiments, the JNK Inhibitor is delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months. The scheduling of such dose regimens can be optimized by the skilled artisan.

In other embodiments, courses of treatment are administered concurrently to a patient, i.e., individual doses of the second active agent are administered separately yet within a time interval such that the JNK Inhibitor can work together with the second active agent. For example, one component can be administered once per week in combination with the other components that can be administered once every two weeks or once every three weeks. In other words, the dosing regimens are carried out concurrently even if the therapeutics are not administered simultaneously or during the same day.

The second active agent can act additively or, more preferably, synergistically with the JNK Inhibitor. In one embodiment, a JNK Inhibitor is administered concurrently with one or more second active agents in the same pharmaceutical composition. In another embodiment, a JNK Inhibitor is administered concurrently with one or more second active agents in separate pharmaceutical compositions. In still another embodiment, a JNK Inhibitor is administered prior to or subsequent to administration of a second active agent. The invention contemplates administration of a JNK Inhibitor and a second active agent by the same or different routes of administration, e.g., oral and parenteral. In certain embodiments, when a JNK Inhibitor is administered concurrently with a second agent that potentially produces adverse side effects including, but not limited to, toxicity, the second active agent can advantageously be administered at a dose that falls below the threshold that the adverse side effect is elicited.

### 4.2.2 Use with Conventional Therapy

The standard methods of chemotherapy, radiation therapy, photodynamic therapy, and surgery are used for treating or managing mesothelioma. Kaiser L R., Semin Thorac Cardiovasc Surg. October; 9(4):383-90, 1997. Intracavitary approaches using targeted cytokines and gene therapy have been tried in patients with mesothelioma using intratumoral gene transfer of recombinant adenovirus (rAd) containing herpes simplex virus thymidine kinase (HSVtk) gene into the pleural space of patients. Id. and Sterman D H, Hematol Oncol Clin North Am. June; 12(3):553-68, 1998.

Certain embodiments of this invention encompass methods of treating or managing asbestos-related diseases or disorders, which comprise administering an effective amount of a JNK Inhibitor in conjunction with (e.g., before, during, or after) conventional therapy including, but not limited to, chemotherapy, surgery, photodynamic therapy, radiation therapy, gene therapy, immunotherapy or other non-drug based therapy presently used to treat or manage the diseases or disorders. The combined use of a JNK Inhibitor and conventional therapy can provide a unique treatment regimen that is unexpectedly effective in certain patients.

As discussed elsewhere herein, the invention encompasses a method of reducing, treating and/or preventing adverse or undesired effects associated with conventional therapy including, but not limited to, chemotherapy, photodynamic therapy, surgery, radiation therapy, gene therapy, and immunotherapy. A JNK Inhibitor and another active agent can be administered to a patient prior to, during, or after the occurrence of the adverse effect associated with conventional therapy. Examples of adverse effects associated with chemotherapy and radiation therapy that can be treated or prevented by this method include, but are not limited to: gastrointestinal toxicity such as, but not limited to, early and late-forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure.

In one embodiment, a JNK Inhibitor is administered in an amount of from about 1 mg to about 5,000 mg per day, from about 1 mg to about 5,000 mg per day, from about 10 mg to about 2,500 mg per day, from about 100 mg to about 800 mg per day, from about 100 mg to about 1,200 mg per day, or from about 25 mg to about 2,500 mg per day orally and daily alone, or in combination with a second active agent disclosed herein (see, e.g., section 4.2.1), prior to, during, or after the use of conventional therapy. In a specific embodiment of this method, an effective amount of a JNK Inhibitor is administered to a patient with mesothelioma who was previously treated with radiotherapy.

In one embodiment of this method, an effective amount of a JNK Inhibitor is administered to a patient with an asbestos-related disease or disorder in combination with trimodality therapy. Trimodality therapy involves a combination of three standard strategies of surgery, chemotherapy, and radiation therapy. In one embodiment of this method, extrapleural pneumonectomy is followed by a combination of chemotherapy using a JNK Inhibitor and radiotherapy. In another embodiment of the trimodality treatment, a JNK Inhibitor is administered in combination with a different chemotherapeutic regimen including a combination of cyclophosphamide adriamycin/cisplatin, carboplatin/paclitaxel, or cisplatin/methotrexate/vinblastine.

### 4.2.3 Cycling Therapy

In certain embodiments, a JNK Inhibitor is cyclically administered to a patient. Cycling therapy involves the administration of a JNK Inhibitor for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment. Consequently, in one specific embodiment of the invention, a JNK Inhibitor is administered daily in a single or divided doses in a four to six week cycle with a rest period of about a week or two weeks. Typically, the number of cycles during which the combinatorial treatment is administered to a patient will be from about one to about 24 cycles, more typically from about two to about 16 cycles, and even more typically from about four to about six cycles. The invention further allows the frequency, number, and length of dosing cycles to be increased. Thus, a specific embodiment of the invention encompasses the administration of a JNK Inhibitor for more cycles than are typical when it is administered alone. In another specific embodiment of the invention, a JNK Inhibitor is administered for a greater number of cycles that would typically cause dose-limiting toxicity in a patient to whom a second active agent is not also being administered.

In one embodiment, a JNK Inhibitor is administered daily and continuously for three or four weeks at a dose of from about 400 to about 1,200 mg/d followed by a break of one or two weeks in a four or six week cycle.

In another embodiment of the invention, a JNK Inhibitor and a second active agent are administered orally, with administration of a JNK Inhibitor occurring 30 to 60 minutes prior to a second active agent, during a cycle of four to six weeks.

In another embodiment, a JNK Inhibitor is administered with cisplatin in an amount of 100 mg/m² on day 1 and gemcitabine in an amount of 1000 mg/m² intravenously on days 1, 8, and day 15 of a 28-day cycle for 6 cycles.

### 4.3 Pharmaceutical Compositions

The compositions comprising a JNK Inhibitor include bulk-drug compositions useful in the manufacture of pharmaceutical compositions (e.g., impure or non-sterile compositions) and pharmaceutical compositions (i.e., compositions that are suitable for administration to a patient) which can be used in the preparation of unit dosage forms. Such compositions optionally comprise a prophylactically or therapeutically effective amount of a prophylactic and/or therapeutic agent disclosed herein or a combination of those agents and a pharmaceutically acceptable vehicle, carrier or excipient. Preferably, compositions of the invention comprise a prophylactically or therapeutically effective amount of JNK Inhibitor and a second active agent, and a pharmaceutically acceptable vehicle, carrier or excipient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which a JNK Inhibitor is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a patient, the pharmaceutically acceptable vehicles are preferably sterile. Water can be the vehicle when the JNK Inhibitor is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propyleneglycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E.W. Martin.

In a preferred embodiment, the JNK Inhibitor and optionally another therapeutic or prophylactic agent are formulated in accordance with routine procedures as pharmaceutical compositions adapted for intravenous administration to human beings. Typically, JNK Inhibitors for intravenous administration are solutions in sterile isotonic aqueous buffer. Where necessary, the compositions can also include a solubilizing agent. Compositions for intravenous administration can optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the JNK Inhibitor is to be administered by infusion, it can be dispensed, for example, with an infusion bottle containing sterile pharmaceutical grade water or saline. Where the JNK Inhibitor is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for an orally administered JNK Inhibitor. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, and the like. Such vehicles are preferably of pharmaceutical grade.

Further, the effect of the JNK Inhibitor can be delayed or prolonged by proper formulation. For example, a slowly soluble pellet of the JNK Inhibitor can be prepared and incorporated in a tablet or capsule. The technique can be improved by making pellets of several different dissolution rates and filling capsules with a mixture of the pellets. Tablets or capsules can be coated with a film which resists dissolution for a predictable period of time. Even the parenteral preparations can be made long-acting, by dissolving or suspending the compound in oily or emulsified vehicles which allow it to disperse only slowly in the serum.

### 4.4 Formulations

Pharmaceutical compositions for use in accordance with the present invention can be formulated in conventional manner using one or more physiologically acceptable vehicles, carriers or excipients.

Thus, the JNK Inhibitor and optionally a second active agent, and their physiologically acceptable salts and solvates, can be formulated into pharmaceutical compositions for administration by inhalation or insufflation (either through the mouth or the nose) or oral, parenteral or mucosol (such as buccal, vaginal, rectal, sublingual) administration. In one embodiment, local or systemic parenteral administration is used.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the JNK Inhibitor.

For buccal administration the pharmaceutical compositions can take the form of tablets or lozenges formulated in conventional manner.

For administration by inhalation, the pharmaceutical compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The pharmaceutical compositions can be formulated for parenteral administration by injection, e.g., by bolus injection or continuous infusion. Formulations for injection can be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The pharmaceutical compositions can also be formulated in rectal compositions such as suppositories or retention enemas, e.g., containing conventional suppository bases such as cocoa butter or other glycerides.

In addition to the formulations described previously, the pharmaceutical compositions can also be formulated as a depot preparation. Such long acting formulations can be administered by implantation (for example subcutaneously or intramuscularly) or by intramuscular injection. Thus, for example, the pharmaceutical compositions can be formulated with suitable polymeric or hydrophobic materials (for example as an emulsion in an acceptable oil) or ion exchange resins, or as sparingly soluble derivatives, for example, as a sparingly soluble salt.

The invention also provides that a pharmaceutical composition can be packaged in a hermetically sealed container such as an ampoule or sachette indicating the quantity. In one embodiment, the pharmaceutical composition is supplied as a dry sterilized lyophilized powder or water free concentrate in a hermetically sealed container and can be reconstituted, e.g., with water or saline to the appropriate concentration for administration to a patient.

The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

In certain preferred embodiments, the pack or dispenser contains one or more unit dosage forms containing no more than the recommended dosage formulation as determined in the Physician's Desk Reference (56^{th} ed. 2002, herein incorporated by reference in its entirety).

### 4.5 Routes of Administration

Methods of administering a JNK Inhibitor and optionally a second active agent include, but are not limited to, parenteral administration (e.g., intradermal, intramuscular, intraperitoneal, intravenous and subcutaneous), epidural, and mucosal (e.g., intranasal, rectal, vaginal, sublingual, buccal or oral routes). In a specific embodiment, the JNK Inhibitor and optionally the second active agent are administered intramuscularly, intravenously, or subcutaneously. The JNK Inhibitor and optionally the second active agent can also be administered by infusion or bolus injection and can be administered together with other biologically active agents. Administration can be local or systemic. The JNK Inhibitor and optionally the second active agent and their physiologically acceptable salts and solvates can also be administered by inhalation or insufflation (either through the mouth or the nose). In one embodiment, local or systemic parenteral administration is used.

In specific embodiments, it can be desirable to administer the JNK Inhibitor locally to the area in need of treatment. This can be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

Pulmonary administration can also be employed, e.g., by use of an inhaler or nebulizer, and formulation with an aerosolizing agent, or via perfusion in a fluorocarbon or synthetic pulmonary surfactant. In certain embodiments, the JNK Inhibitor can be formulated as a suppository, with traditional binders and vehicles such as triglycerides.

In another embodiment, the JNK Inhibitor can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the JNK Inhibitor can be delivered in a controlled release system. In one embodiment, a pump can be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the JNK Inhibitor, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) can be used.

### 4.6 Dosages

The amount of the JNK Inhibitor that is effective in the treatment, prevention or management of CRPS can be determined by standard research techniques. For example, the dosage of the JNK Inhibitor which will be effective in the treatment, prevention or management of CRPS can be determined by administering the JNK Inhibitor to an animal in a model such as, e.g., the animal models known to those skilled in the art. In addition, in vitro assays can optionally be employed to help identify optimal dosage ranges.

Selection of a particular effective dose can be determined (e.g., via clinical trials) by a skilled artisan based upon the consideration of several factors which will be known to one skilled in the art. Such factors include the disease to be treated or prevented, the symptoms involved, the patient's body mass, the patient's immune status and other factors known by the skilled artisan.

The precise dose to be employed in the formulation will also depend on the route of administration, and the seriousness of asbestos-related disease or disorder, and should be decided according to the judgment of the practitioner and each patient's circumstances. Effective doses can be extrapolated from dose-response curves derived from in vitro or animal model test systems.

The dose of a JNK Inhibitor to be administered to a patient, such as a human, is rather widely variable and can be subject to independent judgment. It is often practical to administer the daily dose of a JNK Inhibitor at various hours of the day. However, in any given case, the amount of a JNK Inhibitor administered will depend on such factors as the solubility of the active component, the formulation used, patient condition (such as weight), and/or the route of administration.

The general range of effective amounts of the JNK Inhibitor alone or in combination with a second active agent are from about 0.001 mg/day to about 1000 mg/day, more preferably from about 0.001 mg/day to 750 mg/day, more preferably from about 0.001 mg/day to 500 mg/day, more preferably from about 0.001 mg/day to 250 mg/day, more preferably from about 0.001 mg/day to 100 mg/day, more preferably from about 0.001 mg/day to 75 mg/day, more preferably from about 0.001 mg/day to 50 mg/day, more preferably from about 0.001 mg/day to 25 mg/day, more preferably from about 0.001 mg/day to 10 mg/day, more preferably from about 0.001 mg/day to 1 mg/day. Of course, it is often practical to administer the daily dose of compound in portions, at various hours of the day. However, in any given case, the amount of compound administered will depend on such factors as the solubility of the active component, the formulation used, subject condition (such as weight), and/or the route of administration.

### 4.7 Kits

The invention provides a pharmaceutical pack or kit comprising one or more containers containing a JNK Inhibitor and optionally one or more second active agents useful for the treatment, prevention or management of CRPS. The invention also provides a pharmaceutical pack or kit comprising one or more containers containing one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration; or instructions for the composition's use.

The present invention provides kits that can be used in the above methods. In one embodiment, a kit comprises a JNK Inhibitor, in one or more containers, and optionally one or more second active agents useful for the treatment, prevention or management of CRPS, in one or more additional containers.

### 5. EXAMPLES

The following examples illustrate certain aspects of the invention, but do not limit its scope.

### 5.1 JNK Inhibitor Activity Assays

The ability of a JNK Inhibitor to inhibit JNK and accordingly, to be useful for the treatment, prevention and/or management of an asbestos-related disease or disorder, can be demonstrated using one or more of the following assays.

### 5.1.1 Example

### Biological Activity of 5-amino-anthra(9,1-cd)isothiazol-6-one

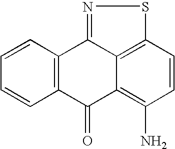 JNK Assay

To 10 μL of 5-amino-anthra(9,1-cd)isothiazol-6-one in 20% DMSO/80% dilution buffer containing of 20 mM HEPES (pH 7.6), 0.1 mM EDTA, 2.5 mM magnesium chloride, 0.004% Triton ×100, 2 μg/mL leupeptin, 20 mM β-glycerolphosphate, 0.1 mM sodium vanadate, and 2 mM DTT in water was added 30 μL of 50-200 ng His6-JNK1, JNK2, or JNK3 in the same dilution buffer. The mixture was pre-incubated for 30 minutes at room temperature. Sixty microliter of 10 μg GST-c-Jun(1-79) in assay buffer consisting of 20 mM HEPES (pH 7.6), 50 mM sodium chloride, 0.1 mM EDTA, 24 mM magnesium chloride, 1 mM DTT, 25 mM PNPP, 0.05% Triton ×100, 11 μM ATP, and 0.5 μCi γ-32P ATP in water was added and the reaction was allowed to proceed for 1 hour at room temperature. The c-Jun phosphorylation was terminated by addition of 150 μL of 12.5% trichloroacetic acid. After 30 minutes, the precipitate was harvested onto a filter plate, diluted with 50 μL of the scintillation fluid and quantified by a counter. The IC₅₀ values were calculated as the concentration of 5-amino-anthra(9,1-cd)isothiazol-6-one at which the c-Jun phosphorylation was reduced to 50% of the control value. Compounds that inhibit JNK preferably have an IC₅₀ value ranging 0.01-10 μM in this assay. 5-Amino-anthra(9,1-cd)isothiazol-6-one has an IC₅₀ according to this assay of 1 μM for JNK2 and 400 nM for JNK3. The measured IC₅₀ value for 5-amino-anthra(9,1-cd)isothiazol-6-one, as measured by the above assay, however, shows some variability due to the limited solubility of 5-amino-anthra(9,1-cd)isothiazol-6-one in aqueous media. Despite the variability, however, the assay consistently does show that 5-amino-anthra(9,1-cd)isothiazol-6-one inhibits JNK. This assay demonstrates that 5-amino-anthra(9,1-cd)isothiazol-6-one, an illustrative JNK Inhibitor, inhibits JNK2 and JNK3 and, accordingly, is useful for the treatment, prevention and/or management of an asbestos-related disease or disorder.

Selectivity For JNK:

5-Amino-anthra(9,1-cd)isothiazol-6-one was also assayed for its inhibitory activity against several protein kinases, listed below, using techniques known to those skilled in art (See, e.g., Protein Phosphorylation, Sefton & Hunter, Eds., Academic Press, pp. 97-367, 1998). The following IC₅₀ values were obtained:

| Enzyme | IC₅₀ |
|---|---|
| p38-2 | >30,000 nM |
| MEK6 | >30,000 nM |
| LKK1 | >30,000 nM |
| IKK2 | >30,000 nM |

This assay shows that 5-amino-anthra(9,1-cd)isothiazol-6-one, an illustrative JNK Inhibitor, selectively inhibits JNK relative to other protein kinases and, accordingly, is a selective JNK Inhibitor. Therefore, 5-amino-anthra(9,1-cd)isothiazol-6-one, an illustrative JNK Inhibitor, is useful for the treatment, prevention and/or management of an asbestos-related disease or disorder.

Jurkat T-Cell IL-2 Production Assay:

Jurkat T cells (clone E6-1) were purchased from the American Type Culture Collection of Manassas, Va. and maintained in growth media consisting of RPMI 1640 medium containing 2 mM L-glutamine (commercially available from Mediatech Inc. of Herndon, Va.), with 10% fetal bovine serum (commercially available from Hyclone Laboratories Inc. of Omaha, Nebr.) and penicillin/streptomycin. All cells were cultured at 37° C. in 95% air and 5% CO₂. Cells were plated at a density of 0.2×10⁶ cells per well in 200 μL of media. Compound stock (20 mM) was diluted in growth media and added to each well as a 10× concentrated solution in a volume of 25 μL, mixed, and allowed to pre-incubate with cells for 30 minutes. The compound vehicle (dimethylsulfoxide) was maintained at a final concentration of 0.5% in all samples. After 30 minutes the cells were activated with PMA (phorbol myristate acetate, final concentration 50 ng/mL) and PHA (phytohemagglutinin, final concentration 2 μg/mL). PMA and PHA were added as a 10× concentrated solution made up in growth media and added in a volume of 25 μL per well. Cell plates were cultured for 10 hours. Cells were pelleted by centrifugation and the media removed and stored at −20° C. Media aliquots are analyzed by sandwich ELISA for the presence of IL-2 as per the manufacturers instructions (Endogen Inc. of Woburn, Mass.). The IC₅₀ values were calculated as the concentration of 5-amino-anthra(9,1-cd)isothiazol-6-one at which the IL-2 production was reduced to 50% of the control value. Compounds that inhibit JNK preferably have an IC₅₀ value ranging from 0.1-30 μM in this assay. 5-Amino-anthra(9,1-cd)isothiazol-6-one has an IC₅₀of 30 μM. The measured IC₅₀ value for 5-amino-anthra(9,1-cd)isothiazol-6-one, as measured by the above assay, however, shows some variability due to the limited solubility of 5-amino-anthra(9,1-cd)isothiazol-6-one in aqueous media. Despite the variability, however, the assay consistently does show that 5-amino-anthra(9,1-cd)isothiazol-6-one inhibits JNK.

This assay shows that 5-amino-anthra(9,1-cd)isothiazol-6-one, an illustrative JNK Inhibitor, inhibits IL-2 production in Jurkat T-cells and accordingly inhibits JNK. Therefore, 5-amino-anthra(9,1-cd)isothiazol-6-one, an illustrative JNK Inhibitor, is useful for the treatment, prevention and/or management of an asbestos-related disease or disorder.

[³H]Dopamine Cell Culture Assay:

Cultures of dopaminergic neurons were prepared according to a modification of the procedure described by Raymon and Leslie (J. Neurochem. 62:1015-1024, 1994). Time-mated pregnant rats were sacrificed on embyronic day 14-15 (crown rump length 11-12 mm) and the embryos removed by cesarean section. The ventral mesencephalon, containing the dopaminergic neurons, was dissected from each embryo. Tissue pieces from approximately 48 embryos were pooled and dissociated both enzymatically and mechanically. An aliquot from the resulting cell suspension was counted and the cells were plated in high glucose DMEM/F12 culture medium with 10% fetal bovine serum at a density of 1×10⁵ cells/well of a Biocoat poly-D-lysine-coated 96-well plate. The day following plating was considered 1 day in vitro (DIV). Cells were maintained in a stable environment at 37° C., 95% humidity, and 5% CO₂. A partial medium change was performed at 3 DIV. At 7 DIV, cells were treated with the neurotoxin, 6-hydroxydopamine (6-OHDA, 30 μM) in the presence and absence of 5-amino-anthra(9,1-cd)isothiazol-6-one. Cultures were processed for [³H]dopamine uptake 22 hours later.

[³H]Dopamine uptake is used as a measure of the health and integrity of dopaminergic neurons in culture (Prochiantz et al., PNAS 76: 5387-5391, 1979). It was used in these studies to monitor the viability of dopaminergic neurons following exposure to the neurotoxin 6-OHDA. 6-OHDA has been shown to damage dopaminergic neurons both in vitro and in vivo and is used to model the cell death observed in Parkinson's disease (Ungerstedt, U., Eur. J. Pharm., 5 (1968) 107-110 and Hefti et al., Brain Res., 195 (1980) 123-137). Briefly, cells treated with 6-OHDA in the presence and absence of 5-amino-anthra(9,1-cd)isothiazol-6-one were assessed in the uptake assay 22 hrs after exposure to 6-OHDA. Culture medium was removed and replaced with warm phosphate buffered saline (PBS) with calcium and magnesium, 10 μM pargyline, 1 mM ascorbic acid, and 50 nM [³H]dopamine. Cultures were incubated at 37° C. for 20 min. Radioactivity was removed and the cultures were washed 3× with ice cold PBS. To determine the intracellular accumulation of [³H]dopamine, cells were lysed with M-PER detergent and an aliquot was taken for liquid scintillation counting. The measured effect of 5-amino-anthra(9,1-cd) isothiazol-6-one on the intracellular accumulation of [³H]dopamine, as measured by the above assay, however, shows some variability due to the limited solubility of 5-amino-anthra(9,1-cd)isothiazol-6-one in aqueous media. Despite the variability, however, the assay consistently does show that 5-amino-anthra(9,1-cd)isothiazol-6-one protects rat ventral mesencephalan neurons from the toxic effects of 6-OHDA. Accordingly, 5-amino-anthra(9,1-cd)isothiazol-6-one, an illustrative JNK Inhibitor, is useful for the treatment, prevention and/or management of an asbestos-related disease or disorder.

Brain-Blood Plasma Distribution of 5-amino-anthra(9.1-cd)isothiazol-6-one In Vivo

5-Amino-anthra(9,1-cd)isothiazol-6-one was administered intravenously (10 mg/kg) into the veins of Sprague-Dawley rats. After 2 hr, blood samples were obtained from the animals and their vascular systems were perfused with approximately 100 mL of saline to rid their brains of blood. The brains were removed from the animals, weighed, and homogenized in a 50 mL conical tube containing 10 equivalents (w/v) of methanol/saline (1:1) using a Tissue Tearer (Fischer Scientific). The homogenized material was extracted by adding 600 μL of cold methanol to 250 μL of brain homogenate vortexed for 30 sec and subjected to centrifugation for 5 min. After centrifugation, 600 μL of the resulting supernatant was transferred to a clean tube and evaporated at room temperature under reduced pressure to provide a pellet. The resulting pellet was reconstituted in 250 μL of 30% aqueous methanol to provide a brain homogenate analysis sample. A plasma analysis sample was obtained using the brain homogenate analysis sample procedure described above by substituting plasma for brain homogenate. Standard plasma samples and standard brain homogenate samples containing known amounts of 5-amino-anthra(9,1-cd)isothiazol-6-one were also prepared by adding 5 μL of serial dilutions (50:1) of a solution of 5-amino-anthra(9,1-cd)isothiazol-6-one freshly prepared in cold ethanol to 250 μL of control rat plasma (Bioreclamation of Hicksville, N.Y.) or control brain homogenate. The standard plasma samples and standard brain homogenate samples were then subjected to the same extraction by protein precipitation, centrifugation, evaporation, and reconstitution procedure used for the brain homogenate to provide brain homogenate standard analysis samples and plasma standard analysis samples. The brain homogenate analysis samples, plasma analysis samples, and standard analysis samples were analyzed and compared using HPLC by injecting 100 μL of a sample onto a 5 μm C-18 Luna column (4.6 mm×150 mm, commercially available from Phenomenex of Torrance, Calif.) and eluting at 1 mL/min with a linear gradient of 30% aqueous acetonitrile containing 0.1% trifluoroacetic acid to 90% aqueous acetonitrile containing 0.1% trifluoroacetic acid over 8 minutes and holding at 90% aqueous acetonitrile containing 0.1% trifluoroacetic acid for 3 min. with absorbance detection at 450 nm. Recovery of 5-amino-anthra(9,1-cd)isothiazol-6-one was 56±5.7% for plasma and 42±6.2% for the brain. The concentration of 5-amino-anthra(9,1-cd) isothiazol-6-one in the brain and plasma was determined by comparing HPLC chromatograms obtained from the brain homogenate analysis samples and plasma analysis samples to standard curves constructed from analysis of the brain homogenate standard analysis samples and the plasma standard analysis samples, respectively. Results from this study show that 5-amino-anthra(9,1-cd)isothiazol-6-one, following intravenous administration, crosses the blood-brain barrier to a significant extent. In particular, brain-drug concentrations were approximately 65 nmole/g and plasma concentrations were approximately 7 μM at 2 hr post-dose, resulting in a brain-plasma concentration ratio of approximately 9-fold (assuming 1 g of brain tissue is equivalent to 1 mL of plasma). This example shows that 5-amino-anthra(9,1-cd)isothiazol-6-one, an illustrative JNK Inhibitor, has enhanced ability to cross the blood-brain barrier. In addition, this example shows that the JNK Inhibitors, in particular 5-amino-anthra(9,1-cd)isothiazol-6-one, can cross the blood-brain barrier when administered to a patient.

### 5.2 Clinical Studies in Mesothelioma Patients

Clinical trials with the administration of 1-(5-(1H-1,2,4-triazol-5-yl)(1H-indazol-3-yl))-3-(2-piperidylethoxy)benzene and vinorelbine are conducted in patients with malignant mesothelioma and malignant pleural effusion mesothelioma syndrome. Patients receive 1-1000 mg per day, 1-500 mg per day, 1-250 mg per day or 1-100 mg per day of 1-(5-(1H-1,2,4-triazol-5-yl)(1H-indazol-3-yl))-3-(2-piperidylethoxy)benzene for 10, 20, 30, 60, 90, 120 or 200 days. Patients who experience clinical benefit are permitted to continue on treatment.

Other clinical studies are performed using 1-(5-(1H-1,2,4-triazol-5-yl)(1H-indazol-3-yl))-3-(2-piperidylethoxy)benzene in unresectable or relapsed mesothelioma patients that have not responded to conventional therapy. In one embodiment, 1-(5-(1H-1,2,4-triazol-5-yl)(1H-indazol-3-yl))-3-(2-piperidylethoxy)benzene is administered in an amount of 1-1000 mg per day, 1-500 mg per day, 1-250 mg per day or 1-100 mg per day, to the patients for 10, 20, 30, 60, 90, 120 or 200 days. It is understood that other preferred embodiments are when 1-(5-(1H-1,2,4-triazol-5-yl)(1H-indazol-3-yl))-3-(2-piperidylethoxy)benzene is administered at about 75-900 mgs/day or a greater dose, or at about 1.5 to 2.5 times the daily dose every other day. The studies in mesothelioma patients treated with a JNK Inhibitor will show that the drug has therapeutic benefit in this disease.

It will be appreciated that, although specific embodiments of the invention have been described herein for purposes of illustration, the invention described and claimed herein is not to be limited in scope by the specific embodiments herein disclosed. These embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

A number of references have been cited, the entire disclosure of which are incorporated herein by reference in their entirety.

What is claimed is:

1. A method for treating, preventing and/or managing an asbestos-related disease or disorder in a patient, comprising administering to a patient in need thereof an effective amount of a JNK Inhibitor or a pharmaceutically acceptable salt thereof.

2. A method for treating, preventing and/or managing an asbestos-related disease or disorder in a patient, comprising administering to a patient in need thereof an effective amount of a compound having the following formula: 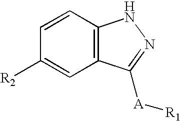
or a pharmaceutically acceptable salt thereof,
wherein:
A is a direct bond, —(CH₂)ₐ—, —(CH₂)_{b}CH═CH(CH₂)_{c}—, or —(CH₂)_{b}C≡C(CH₂)_{c}—;
R₁ is aryl, heteroaryl or heterocycle fused to phenyl, each being optionally substituted with one to four substituents independently selected from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;
a is 1, 2, 3, 4, 5 or 6;
b and c are the same or different at each occurrence independently selected from 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, substituted aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently selected from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

3. A method for treating, preventing and/or managing an asbestos-related disease or disorder in a patient, comprising administering to a patient in need thereof an effective amount of a compound having the following formula: 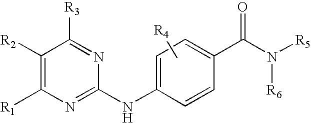
or a pharmaceutically acceptable salt thereof,
wherein:
R₁ is aryl or heteroaryl optionally substituted with one to four substituents independently selected from R₇;
R₂ is hydrogen;
R₃ is hydrogen or lower alkyl;
R₄ represents one to four optional substituents, wherein each substituent is the same or different and independently selected from halogen, hydroxy, lower alkyl and lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀ and R₁₁ are the same or different and at each occurrence independently hydrogen, alkyl, substituted alkyl, aryl, arylalkyl, heterocycle or heterocycloalkyl;
or R₈ and R₉ taken together with the atom or atoms to which they are attached to form a heterocycle;
a and b are the same or different and at each occurrence independently selected from 0, 1, 2, 3 or 4; and
c is at each occurrence 0, 1 or 2.

4. A method for treating, preventing and/or managing an asbestos-related disease or disorder in a patient, comprising administering to a patient in need thereof an effective amount of a compound having the following formula: 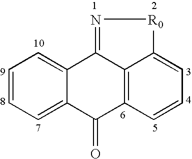
or a pharmaceutically acceptable salt thereof,
wherein R₀ is —O—, —S—, —S(O)—, —S(O)₂—, NH or —CH₂—;
the compound being (i) unsubstituted, (ii) monosubstituted and having a first substituent, or (iii) disubstituted and having a first substituent and a second substituent;
the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by formula (a), (b), (c), (d), (e), or (f): 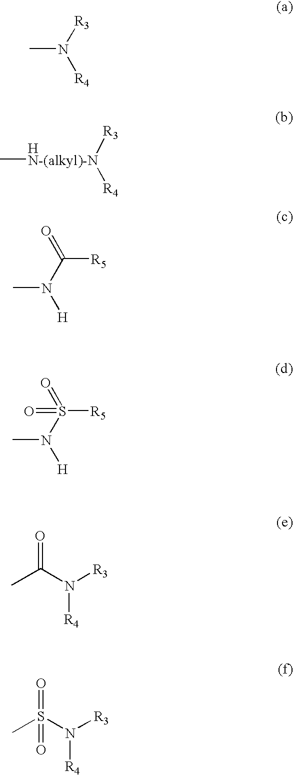
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

5. The method of claim 2 wherein A is a direct bond.

6. The method of claim 2 wherein A is —(CH₂)ₐ—.

7. The method of claim 2 wherein A is —(CH₂)_{b}CH═CH(CH₂)_{c}—.

8. The method of claim 2 wherein A is —(CH₂)_{b}C≡C(CH₂)_{c}—.

9. The method of claim 2 wherein the compound has the following formula: 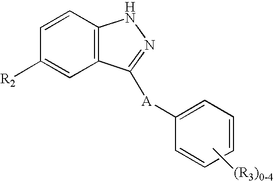
or a pharmaceutically acceptable salt thereof,
wherein:
A is a direct bond, —(CH₂)ₐ—, —(CH₂)_{b}CH═CH(CH₂)_{c}—, or —(CH₂)_{b}C≡C(CH₂)_{c}—;
R₁ is aryl, heteroaryl or heterocycle fused to phenyl, each being optionally substituted with one to four substituents independently selected from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;
a is 1, 2, 3, 4, 5 or 6;
b and c are the same or different and at each occurrence independently selected from 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently selected from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and
R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

10. The method of claim 2 wherein the compound has the following formula: 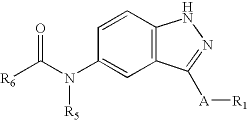
or a pharmaceutically acceptable salt thereof,
wherein:
A is a direct bond, —(CH₂)₁—, —(CH₂)_{b}CH═CH(CH₂)_{c}—, or —(CH₂)_{b}C≡C(CH₂)_{c}—;
R₁ is aryl, heteroaryl or heterocycle fused to phenyl, each being optionally substituted with one to four substituents independently selected from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ —or —(CH₂)_{b}SO₂NR₅R₆;
a is 1, 2, 3, 4, 5 or 6;
b and c are the same or different and at each occurrence independently selected from 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently selected from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and
R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

11. The method of claim 2 wherein the compound has the following formula: 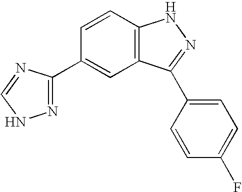
or a pharmaceutically acceptable salt thereof.

12. The method of claim 3, wherein the compound has the following formula: 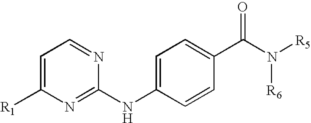
or a pharmaceutically acceptable salt thereof,
wherein:
R₁ is aryl or heteroaryl optionally substituted with one to four substituents independently selected from R₇;
R₂ is hydrogen;
R₃ is hydrogen or lower alkyl;
R₄ represents one to four optional substituents, wherein each substituent is the same or different and independently selected from halogen, hydroxy, lower alkyl and lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;
or R₅ and R6 taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₀, —NR₈C(═O)(CH₂)_{b}OR₉, —NR₈C(═O)(CH₂)_{b}R₉, —(CH₂)_{b}NR₈R₉, or heterocycle fused to phenyl;
R₈, R₉, R₁₀ and R₁₁ are the same or different and at each occurrence independently hydrogen, alkyl, substituted alkyl, aryl, substituted aryl, arylalkyl, heterocycle, heterocycloalkyl;
or R₈ and R₉ taken together with the atom or atoms to which they are attached to form a heterocycle;
a and b are the same or different and at each occurrence independently selected from 0, 1, 2, 3 or 4; and
c is at each occurrence 0, 1 or 2.

13. The method of claim 3, wherein the compound has the following formula: 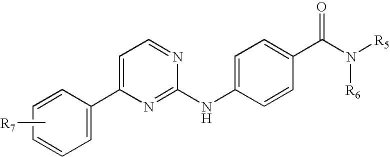
or a pharmaceutically acceptable salt thereof,
wherein:
R₁ is aryl or heteroaryl optionally substituted with one to four substituents independently selected from R₇;
R₂ is hydrogen;
R₃ is hydrogen or lower alkyl;
R₄ represents one to four optional substituents, wherein each substituent is the same or different and independently selected from halogen, hydroxy, lower alkyl and lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(—O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀ and R₁₁ are the same or different and at each occurrence independently hydrogen, alkyl, aryl, arylalkyl, heterocycle, heterocycloalkyl;
or R₈ and R₉ taken together with the atom or atoms to which they are attached to form a heterocycle;
a and b are the same or different and at each occurrence independently selected from 0, 1, 2, 3 or 4; and
c is at each occurrence 0, 1 or 2.

14. The method of claim 3, wherein the compound has the following formula: 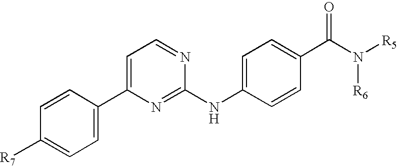
or a pharmaceutically acceptable salt thereof,
wherein:
R₁ is aryl or heteroaryl optionally substituted with one to four substituents independently selected from R₇;
R₂ is hydrogen;
R₃ is hydrogen or lower alkyl;
R₄ represents one to four optional substituents, wherein each substituent is the same or different and independently selected from halogen, hydroxy, lower alkyl and lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)₁NR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;
or R₅ and R₆ taken together with the nitrogen atom to which they are attached to form a heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀ and R₁₁ are the same or different and at each occurrence independently hydrogen, alkyl, substituted alkyl, aryl, arylalkyl, heterocycle, heterocycloalkyl;
or R₈ and R₉ taken together with the atom or atoms to which they are attached to form a heterocycle;
a and b are the same or different and at each occurrence independently selected from 0, 1, 2, 3 or 4; and
c is at each occurrence 0, 1 or 2.

15. The method of claim 4, wherein R₀ is —O—.

16. The method of claim 4, wherein R₀ is —S—.

17. The method of claim 4, wherein R₀ is —(O)—.

18. The method of claim 4, wherein R₀ is —(O)₂—.

19. The method of claim 4, wherein R₀ is NH.

20. The method of claim 4, wherein R₀ is CH₂—.

21. The method of claim 4, wherein the compound has the following formula: 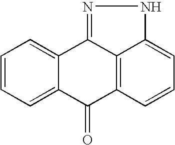
or a pharmaceutically acceptable salt thereof.

22. The method of claim 1, further comprising administering a second active agent.

23. The method of claim 2, further comprising administering a second active agent.

24. The method of claim 3, further comprising administering a second active agent.

25. The method of claim 4, further comprising administering a second active agent.

26. The method of claim 22, wherein the second active agent is an anti-cancer agent, antibiotic, anti-inflammatory agent, steroid, immunomodulatory agent, cytokine, immunosuppressive agent, an IMiD®, a SelCID® or a combination thereof.

27. The method of claim 23, wherein the second active agent is anthracycline, platinum, alkylating agent, interferon, oblimersen, cisplatinum, cyclophosphamide, irinotecan, topotecan, temozolomide, temodar, carboplatin, procarbazine, gliadel, tamoxifen, methotrexate, taxotere, capecitabine, cisplatin, thiotepa, fludarabine, liposomal daunorubicin, cytarabine, doxetaxol, pacilitaxel, vinblastine, GM-CSF, IL-2, dacarbazine, vinorelbine, zoledronic acid, palmitronate, biaxin, busulphan, prednisone, bisphosphonate, arsenic trioxide, vincristine, doxorubicin, paclitaxel, ganciclovir, adriamycin, bleomycin, hyaluronidase, mitomycin C, mepacrine, thiotepa, tetracycline, thalidomide or gemcitabine.

28. The method of claim 1, wherein the disease or disorder is mesothelioma, asbestosis, pleural effusion, pleural plaque, pleural calcification, diffuse pleural thickening, round atelectasis, or bronchogenic carcinoma.

29. A method of treating, preventing or managing an asbestos-related disease or disorder, which comprises administering to a patient in need of such treatment, prevention or management an effective amount of a JNK Inhibitor, or a pharmaceutically acceptable salt thereof, before, during or after chemotherapy, photodynamic therapy, surgery, radiation therapy, gene therapy, or immunotherapy.
